SEQUENCE LISTING (8 sequences)

SEQ ID NO: 1 - DNA, 23 bp, Artificial Sequence
  Description of Artificial Sequence Synthetic primer
atcagggccaagacatagagatg

SEQ ID NO: 2 - DNA, 20 bp, Artificial Sequence
  Description of Artificial Sequence Synthetic primer
gccaatgcaacttggacgtt

SEQ ID NO: 3 - DNA, 24 bp, Artificial Sequence
  Description of Artificial Sequence Synthetic primer
ttcacatgtcccagcactaccaga

SEQ ID NO: 4 - DNA, 26 bp, Artificial Sequence
  Description of Artificial Sequence Synthetic primer
tcacatgtgtgagaggggcagtgtgc

SEQ ID NO: 5 - DNA, 24 bp, Artificial Sequence
  Description of Artificial Sequence Synthetic primer
acccatccttcctgcccgatcaga

SEQ ID NO: 6 - DNA, 24 bp, Artificial Sequence
  Description of Artificial Sequence Synthetic primer
ttggtaatggagcggcgggacttg

SEQ ID NO: 7 - DNA, 20 bp, Artificial Sequence
  Description of Artificial Sequence Synthetic primer
gccaggaggtcttcgctgta

SEQ ID NO: 8 - DNA, 20 bp, Artificial Sequence
  Description of Artificial Sequence Synthetic primer
aatgcacggctagggtcaaa

(19) United States
(12) Patent Application Publication
(10) Pub. No.: US 2017/0283777 A1
(43) Pub. Date: Oct. 5, 2017

(54) MAMMALIAN CHIMERIC COMPLEMENTATION

(71) Applicant: Salk Institute for Biological Studies, La Jolla, CA (US)

(72) Inventors: Juan Carlos IZPISUA BELMONTE, La Jolla, CA (US); Jun WU, La Jolla, CA (US)

(21) Appl. No.: 15/468,006

(22) Filed: Mar. 23, 2017

Related U.S. Application Data
(60) Provisional application No. 62/313,623, filed on Mar. 25, 2016.

(51) Int. Cl. C12N 5/071; C12N 15/85

(52) U.S. Cl. CPC C12N 5/0697; C12N 15/85; C12N 2800/108

(57) ABSTRACT

Disclosed herein are chimeric mammals, such as a chimeric mammal comprising cells derived from at least a first mammal and a second mammal, wherein the cells from the first mammal comprise a genetic modification at one or more loci and the cells from the second mammal form at least one organ or tissue, wherein the first and second mammals are different species.

### CROSS-REFERENCE

This application claims the benefit of priority from U.S. Provisional Patent Application No. 62/313,623, filed Mar. 25, 2016, which is herein incorporated by reference in its entirety.

### BACKGROUND OF THE INVENTION

The successful derivation of human pluripotent stem cells (hPSCs), including embryonic stem cells (ESCs) from pre-implantation human embryos and induced pluripotent stem cells (iPSCs) from somatic cells through cellular reprogramming, provides an opportunity that has revolutionized the way human development is studied and is also considered the herald of a new age in regenerative medicine. hPSCs are not only conducive to a better understanding specific functional and dysfunctional aspects of human biology, but also have potential to offer an inexhaustible supply of cells, tissues and organs for future replacement therapies.

### SUMMARY OF THE INVENTION

Contemplated herein are chimeric mammals and organs derived from chimeric mammals cultured using methods of mammalian chimeric complementation described herein. Such chimeric mammals and organs derived from chimeric mammals provide novel methods and therapeutics as described in the present disclosure.

In certain aspects, there are provided, chimeric mammals comprising cells derived from at least a first mammal and a second mammal, wherein the cells from the first mammal comprise cells derived from a blastocyst comprising a genetic modification at one or more loci and the cells from the second mammal comprise cells derived from a naïve-like induced pluripotent stem cell (naïve-like iPSC) and form at least one organ or tissue, wherein the first and second mammal are different species. In some embodiments, the first mammal is selected from the group consisting of mouse, rat, rabbit, guinea pig, cow, pig, horse, goat, and sheep. In some embodiments, the first mammal is a cow or a pig. In some embodiments, the second mammal is selected from the group consisting of mouse, rat, rabbit, guinea pig, human, cow, pig, horse, goat, and sheep. In some embodiments, the second mammal is a human. In some embodiments, the chimeric mammal has a percentage chimerism as a ratio between the second mammal and the first mammal of less than 10%. In some embodiments, the organ or tissue comprises at least one of the group consisting of liver, kidney, pancreas, hematopoietic stem cells, spleen, bone marrow, heart, lung, skin, cornea, eye, spinal cord, uterus, intestine, heart valve, bone, cartilage, tendon, ligament, lymphatic vessel, and blood vessel. In some embodiments, the organ or tissue is chimeric. In some embodiments, the organ or tissue has a percentage chimerism as a ratio between the second mammal and the first mammal of at least 10%. In some embodiments, the organ or tissue has a percentage chimerism as a ratio between the second mammal and the first mammal of at least 50%. In some embodiments, the organ or tissue has a percentage chimerism as a ratio between the second mammal and the first mammal of at least 90% to 99%. In some embodiments, the genetic modification disables organogenesis of an organ or tissue. In some embodiments, the genetic modification at one or more loci inactivates at least one gene selected from the group consisting of FAH, NKX2.5, TBX5, MEF2C, Osr1, Lhx1, Pax2, Pax8, Wt1, Hox11, Eya1, Six2, Sal11, Etv2, Trox1, Ronx-1, Scl/Tal-1, Lmo-2, Tel, Tek, Sox9, Scleraxis, Pax6, and Rx. In some embodiments, the iPSC is derived from a hiPSC type culture condition selected from 2iLD, NHSM, 4i, and FAC. In some embodiments, the iPSC is a naïve-like iPSC or naïve-like hiPSC. In some embodiments, the genetic modification at one or more loci inactivates Pdx1 and the cells from the second mammal form at least a portion of a pancreas. In some embodiments, the genetic modification at one or more loci inactivates one or more genes including Runx-1, Scl/Tal-1, Lmo-2, Tel, and/or Tek and the cells from the second mammal form at least a portion of a blood vessel. In some embodiments, the genetic modification at one or more loci inactivates FAH and the cells from the second mammal form at least a portion of a liver. In some embodiments, the genetic modification at one or more loci inactivates one or more genes including Osr1, Lhx1, Pax2, Pax8, Wt1, Hox11, Eya1, Six2, and/or Sal11 and the cells from the second mammal form at least a portion of a kidney. In some embodiments, the genetic modification at one or more loci inactivates one or more genes including Nkx2.5, Tbx5, and/or Mef2c and the cells from the second mammal form at least a portion of a heart. In some embodiments, the genetic modification at one or more loci inactivates one or more genes including Pax6 and/or Rx1 and the cells from the second mammal form at least a portion of an eye. In some embodiments, the genetic modification at one or more loci inactivates one or more genes including Sox9 and/or Scleraxis and the cells from the second mammal form at least a portion of a cartilage tissue. In some embodiments, the genetic modification at one or more loci inactivates one or more genes including Etv2 and/or Prox1 and the cells from the second mammal form at least a portion of an endothelial or lymphatic vessel.

In certain aspects, there are provided, mammalian organs or tissues isolated from a chimeric mammal comprising cells derived from at least a first mammal and a second mammal, wherein the organ or tissue is derived from an induced pluripotent stem cell (iPSC) cell from the second mammal, wherein the first and second mammal are different species. In some embodiments, the organ or tissue comprises at least one of the group consisting of liver, kidney, pancreas, hematopoietic stem cells, spleen, bone marrow, heart, lung, skin, cornea, eye, spinal cord, uterus, intestine, heart valve, bone, cartilage, tendon, ligament, lymphatic vessel, and blood vessel. In some embodiments, the second mammal is selected from the group consisting of mouse, rat, rabbit, guinea pig, human, cow, pig, horse, goat, and sheep. In some embodiments, the second mammal is a human. In some embodiments, the first mammal is selected from the group consisting of mouse, rat, rabbit, guinea pig, cow, pig, horse, goat, and sheep. In some embodiments, the first mammal is a cow or a pig. In some embodiments, cells of the first mammal comprise a genetic modification at one or more loci. In some embodiments, the genetic modification inactivates at least one gene selected from the group consisting of FAH, NKX2.5, TBX5, MEF2C, Osr1, Lhx1, Pax2, Pax8, Wt1, Hox11, Eya1, Six2, Sal11, Etv2, Trox1, Ronx-1, Scl/Tal-1, Lmo-2, Tel, Tek, Sox9, Scleraxis, Pax6, and Rx. In some embodiments, the iPSC is derived from a hiPSC type culture condition selected from 2iLD, NHSM, 4i, and FAC. In some embodiments, the iPSC is a naïve-like iPSC or naöve-like hiPSC. In some embodiments, the first mammal comprises a genetic modification that inactivates Pdx1 and the cells from the second mammal form at least a portion of a pancreas. In some embodiments, the first mammal comprises genetic modifications that inactivate one or more gens including Runx-1, SCl/Tal-1, Lmo-2, Tel, and/or Tek and the cells from the second mammal form at least a portion of a blood vessel or hematopoietic cell. In some embodiments, the first mammal comprises genetic modifications that inactivate FAH and the cells from the second mammal form at least a portion of a liver. In some embodiments, the first mammal comprises genetic modifications that inactivate one or more genes including Osr1, Lhx1, Pax2, Pax8, Wt1, Hox11, Eya1, Six2, and/or Sal11 and the cells from the second mammal form at least a portion of a kidney. In some embodiments, the first mammal comprises genetic modifications that inactivate one or more genes including Nkx2.5, Tbx5, and/or Mef2c and the cells from the second mammal form at least a portion of a heart. In some embodiments, the first mammal comprises genetic modifications that inactivate one or more genes including Pax6 and/or Rx and the cells from the second mammal form at least a portion of an eye. In some embodiments, the first mammal comprises genetic modifications that inactivate one or more genes including Sox9 and/or Scleraxis and the cells from the second mammal form at least a portion of a cartilage tissue. In some embodiments, the first mammal comprises genetic modifications that inactivate one or more genes including Etv2 and/or Prox1 and the cells from the second mammal form at least a portion of an endothelial or lymphatic vessel.

In certain aspects, there are provided methods of culturing mammalian organs or tissues comprising: a) injecting a blastocyst of a first mammal with at least one iPSC from a second mammal to form a chimeric blastocyst; b) transferring the chimeric blastocyst into a pseudo-pregnant third mammal; and c) obtaining a chimeric mammal comprising the mammalian organ or tissue; wherein the first and second mammal are different species, and wherein the first mammal is genetically modified at one or more loci and the organ or tissue is derived from iPSC from the second mammal. In some embodiments, between 1 and 15 iPSCs from the second mammal are injected into the blastocyst of the first mammal In some embodiments, the blastocyst of the first mammal is injected between days 4 and 7 post-fertilization. In some embodiments, the chimeric blastocyst is transferred 2 to 30 hours after injection. In some embodiments, the second mammal is selected from the group consisting of mouse, rat, rabbit, guinea pig, human, cow, pig, horse, goat, and sheep. In some embodiments, the second mammal is a human. In some embodiments, the first mammal and the third mammal are selected from the group consisting of mouse, rat, rabbit, guinea pig, cow, pig, horse, goat, and sheep. In some embodiments, the first mammal and the third mammal are a cow or a pig. In some embodiments, the first mammal and the third mammal are the same species. In some embodiments, the blastocyst of the first mammal is genetically modified at a locus by a method comprising contacting a zygote of the first mammal with a CRISPR/CAS reagent that inactivates at least one gene at the locus. In some embodiments, the second mammal is genetically modified at a locus that inactivates at least one gene selected from the group consisting of FAH, NKX2.5, TBX5, MEF2C, Osr1, Lhx1, Pax2, Pax8, Wt1, Hox11, Eya1, Six2, Sal11, Etv2, Trox1, Ronx-1, Scl/Tal-1, Lmo-2, Tel, Tek, Sox9, Scleraxis, Pax6, and Rx. In some embodiments, the organ or tissue is selected from the group consisting of liver, kidney, pancreas, hematopoietic stem cells, spleen, bone marrow, heart, lung, skin, cornea, eye, spinal cord, uterus, intestine, heart valve, bone, cartilage, tendon, ligament, lymphatic vessel, and blood vessel. In some embodiments, the iPSC is derived from a hiPSC type culture condition selected from 2iLD, NHSM, 4i, and FAC. In some embodiments, the iPSC is a naïve-like iPSC or naïve-like hiPSC. In some embodiments, the first mammal comprises a genetic modification that inactivates Pdx1 and the cells from the second mammal form at least a portion of a pancreas. In some embodiments, the first mammal comprises genetic modifications that inactivate one or more genes including Runx-1, Scl/Tal-1, Lmo-2, Tel, and/or Tek and the cells from the second mammal form at least a portion of a blood vessel. In some embodiments, the first mammal comprises genetic modifications that inactivate FAH and the cells from the second mammal form at least a portion of a liver. In some embodiments, the first mammal comprises genetic modifications that inactivate one or more genes including Osr1, Lhx1, Pax2, Pax8, Wt1, Hox11, Eya1, Six2, and/or Sal11 and the cells from the second mammal form at least a portion of a kidney. In some embodiments, the first mammal comprises genetic modifications that inactivate one or more genes including Nkx2.5, Tbx5, and/or Mef2c and the cells from the second mammal form at least a portion of a heart. In some embodiments, the first mammal comprises genetic modifications that inactivate one or more genes including Pax6 and/or Rx1 and the cells from the second mammal form at least a portion of an eye. In some embodiments, the first mammal comprises genetic modifications that inactivate one or more genes including Sox9 and/or Scleraxis and the cells from the second mammal form at least a portion of a cartilage tissue. In some embodiments, the first mammal comprises genetic modifications that inactivate one or more genes including Etv2 and/or Prox1 and the cells from the second mammal form at least a portion of an endothelial or lymphatic vessel.

Also provided herein, in certain aspects, are methods of testing the safety and efficacy of a drug comprising administering the drug to the chimeric mammal according to any one of claims 1 to 24 and measuring toxicity or therapeutic benefit in the chimeric mammal. In some embodiments, the toxicity comprises at least one of body weight, food consumption, histopathology, immunogenicity, autoimmunity, genotoxicity, nephropathy, hepatopathology, neuropathology, and carcinogenesis. In some embodiments, the therapeutic benefit comprises an intended effect of the drug administered.

In additional aspects, there are provided, methods of testing the safety and efficacy of a drug comprising contacting the drug to the mammalian organ or tissue of any one of claims 25 to 42 and measuring toxicity and in vitro therapeutic benefit. In some embodiments, the toxicity comprises at least one of cellular apoptosis, histopathology, and genotoxicity. In some embodiments, the in vitro therapeutic benefit comprises an intended effect of the drug administered.

In further aspects, there are provided, methods of making an induced pluripotent stem cell (iPSC). Some such methods comprise transfecting a population of human foreskin fibroblasts (HFF) with at least three episomal vectors comprising pCXLE-hOCT3/4-shp53-F, pCXLE-hSK, and pCXLE-hUL; contacting the transfected population of HFFs with a population of mitotically inactivated mouse embryonic fibroblasts (MEFs); contacting the population of HFFs and the population of MEFs with an FAC medium comprising a base media, an FGF, an Activin, and a WNT activator until at least one colony comprising an iPSC has formed; and transferring the colony comprising an iPSC to a fresh population of mitotically inactivated MEFs. In some embodiments, the base media comprises DMEM/F12, Neurobasal medium, N2 supplement, B27 supplement, GlutaMax, non-essential amino acids, beta-mercaptoethanol, and antibiotics. In some embodiments, the FGF comprises an FGF2. In some embodiments, the Activin comprises an Activin A. In some embodiments, the WNT activator comprises a GSK inhibitor. In some embodiments, the GSK inhibitor comprises CHIR99021. In some embodiments, the HFF are transfected with pCXLE-EGFP. In some embodiments, the FAC medium comprises bovine serum albumin

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of a strategy for the generation of naïve-like hiPSCs (human induced pluripotent stem cells) using different naïve culture conditions.

FIGS. 2A-2F show interspecies human-cattle chimeric blastocyst formation with naïve-like hiPSCs.

FIG. 2A shows a schematic of protocol for production of bovine blastocysts obtained from in vitro fertilization (IVF) and subsequently used for laser-assisted blastocyst injection of naïve-like hiPSCs. After injection, bovine blastocysts were cultured in vitro for 2 days before fixation and analyzed by immunostaining with HuNu and SOX2 antibodies. Criteria to evaluate the human cell survival, degree and efficiency of ICM incorporation were shown on the right.

FIG. 2B shows average number of cells remaining in the bovine blastocyst after injection of 10 hiPSCs followed by 2 days embryo culture.

FIG. 2C shows average number of hiPSCs integrated into the bovine ICMs.

FIG. 2D shows percentage of blastocysts with human iPSCs residing in the bovine ICMs.

FIG. 2E shows percentage of SOX2+ cells found in the bovine ICMs.

FIG. 2F shows percentage of human cells residing in the bovine ICM that remained SOX2+.

FIGS. 3A-3F show interspecies human-pig chimeric blastocyst formation with naïve-like hiPSCs

FIG. 3A shows a schematic of protocol for production of porcine blastocysts obtained from parthenogenesis and subsequently used for laser-assisted blastocyst injection of naïve-like hiPSCs. After injection, porcine blastocysts were cultured in vitro for 2 days before fixation and analyzed by immunostaining with Hunu and SOX2 antibodies. Criteria to evaluate the human cell survival, degree and efficiency of ICM incorporation were shown on the right.

FIG. 3B shows average number of hiPSCs remaining in the porcine blastocyst after injection of 10 hiPSCs followed by 2 days embryo culture.

FIG. 3C shows average number of hiPSCs integrated into the porcine ICMs.

FIG. 3D shows percentage of blastocysts with human iPSCs residing in the porcine ICMs.

FIG. 3E shows percentage of SOX2+ cells found in the porcine ICMs.

FIG. 3F shows percentage of cells residing in the porcine ICM that remained SOX2+.

FIGS. 4A-4G show a comparison of morula versus blastocyst injection of hiPSCs in bovine hosts

FIG. 4A shows schematic drawing showing the steps taken to compare morula and blastocyst injection of 4i-hiPSCs and NHSM-hiPSCs to two large animal hosts: pig and cattle.

FIG. 4B shows summary of the comparison between morula versus blastocyst injections of 4i-hiPSCs and NHSM-hiPSCs to bovine embryos.

FIG. 4C shows average number of hiPSCs remaining in the bovine blastocyst after injection of 10 hiPSCs to each morula followed by 4 days of embryo culture.

FIG. 4D shows average number of hiPSCs integrated into the bovine ICMs following morula injections and 4 days of embryo culture.

FIG. 4E shows percentage of blastocysts with human iPSCs resides in the bovine ICMs following morula injections and 4 days of embryo culture.

FIG. 4F shows percentage of SOX2+ cells found in the bovine ICM following morula injections and 4 days of embryo culture.

FIG. 4G shows percentage of hiPSCs residing in the bovine ICM that remained SOX2+ following morula injections and 4 days of embryo culture.

FIGS. 5A-5F show generation of post-implantation human-pig chimeric embryos using naïve-like hiPSCs

FIG. 5A shows a schematic drawing illustrating the procedures involved in generation and analysis of post-implantation chimeric embryos with naïve-like hiPSCs.

FIG. 5B shows a summary of the recovered embryos between days 24 to 28 of pregnancy.

FIG. 5C shows a bar graph showing proportions of normal-sized versus small-sized embryos generated from different types of naïve-like hiPSCs.

FIG. 5D shows a bar graph showing among positive embryos the proportion of normal-sized versus small-sized embryos generated from different types of naïve-like hiPSCs.

FIG. 5E shows a bar graph showing among normal-sized embryos the proportion of positive versus negative embryos generated from different types of naïve-like hiPSCs.

FIG. 5F shows a bar graph showing among small-sized embryos the proportion of positive versus negative embryos generated from different types of naïve-like hiPSCs.

FIGS. 6A-6C show characterization of transgene-free hiPSCs by real-time qPCR.

FIG. 6A shows real-time qPCR analyses of primed HFF-iPSCs generated using episomal vectors with primers of EBNA, SOX2 and KLF4 (primer sequences listed in FIG. 6B). H9 ESCs were used as negative control. HFFs 6 days after nucleofection with episomal factors were included as a positive control.

FIG. 6B shows qPCR primers used in FIG. 6A. FBXO15 was used for internal control. Listed sequences are SEQ ID NO: 1-8 in order of appearance.

FIG. 6C shows a summary of the chimeric contribution of NHSM-hiPSCs to post-implantation E6.5 mouse embryos.

FIGS. 7A-7C show ICM incorporation of naïve-like hiPSCs to porcine blastocysts

FIG. 7A shows comparison between pig and cattle: percentage of blastocysts with cells in porcine ICM.

FIG. 7B shows comparison between pig and cattle: percentage of SOX2+ cells in porcine ICM.

FIG. 7C shows comparison between pig and cattle: percentage of cells found in porcine ICM that are SOX2+.

FIGS. 8A-8I show comparison of morula versus blastocyst injection in porcine hosts

FIG. 8A shows a summary of morula versus blastocyst injections of 4i-hiPSCs and NHSM-hiPSCs in porcine embryos.

FIG. 8B shows average number of cells remained in the porcine blastocyst after morulae injection and embryo culture.

FIG. 8C shows average number of hiPSCs integrated into the porcine ICMs after morula injections and embryo culture.

FIG. 8D shows percentage of blastocysts with human iPSCs in the ICMs following morula injections and embryo culture.

FIG. 8E shows percentage of SOX2+ cells in the ICMs following morula injections and embryo culture.

FIG. 8F shows percentage of hiPSCs inside the ICMs and SOX2+ following morula injections and embryo culture.

FIG. 8G shows a comparison between pig and cattle: percentage of blastocyst with hiPSCs incorporated into the ICM following either morula or blastocyst injection.

FIG. 8H shows a comparison between pig and cattle: percentage of SOX2+ hiPSCs in the ICM following either morula or blastocyst injection.

FIG. 8I shows a comparison between pig and cattle: percentage of hiPSCs inside the ICMs and SOX2+ following either morula or blastocyst injection.

FIGS. 9A-9B show a summary of embryo transfer experiments

FIG. 9A shows a summary of embryo collections and embryo culture for the generation of blastocysts for hiPSCs injections

FIG. 9B shows a summary of hiPSCs blastocyst injections and embryo transfer for the generation of chimeric human-pig post-implantation embryos.

### DETAILED DESCRIPTION OF THE INVENTION

Applicants disclose herein, a first ever interspecies chimera formation from naïve-like hPSCs. Using large animal hosts constitutes a novel in vivo strategy for human tissue generation, drug screening, and disease modeling. Described herein is the discovery of a novel type of naïve-like hiPSCs, developed using novel culture conditions, for chimeric contribution in two large animal species: pig and cattle. Data herein show that different kinds of naïve-like hiPSCs integrate into the ICM and pre-implantation host epiblasts. Moreover, upon embryo transfer to surrogate sows, naïve-like hiPSCs are able to contribute to post-implantation chimeric pig embryos and differentiate into cells representative of the three human germ lineages: ectoderm, mesoderm and endoderm.

Applicant provides a first-ever creation of chimeric mammals made from human naïve-like induced pluripotent stem cells and mammalian host blastocyts from large mammals such as pig and cow using novel methods described herein. Previously, cell pluripotency has been captured in vitro at different states and developmental potentials, ranging from a naïve state able to generate every cell of an adult organism, e.g., mouse embryonic stem cells (mESCs), to a more developmentally advanced primed state, e.g., mouse epiblast stem cells (mEpiSCs). Although primed PSCs, in some cases, give rise to cells of all three germ layers, they exhibit characteristics of the peri-gastrulating epiblast and presumably harbor a more restricted pluripotency program. Several lines of evidence indicate that conventional hPSCs differ from mESCs and more closely resemble mEpiSCs, leading to the notion that hPSCs are in a primed pluripotent state. This consequently fueled the quest for culture conditions that stabilize hPSCs in a more naïve state. Recent reports have demonstrated the successful generation of naïve-like hPSCs either through de novo derivation, conversion from primed cells, or through cellular reprogramming. Among others, establishment of naïve-like hPSCs culture conditions has already provided some practical and experimental advantages, including high single cell cloning efficiency and facile genome editing.

Previous reports of interspecies chimeric mammals used human region selective pluripotent stem cells (rsPSCs) or conventional human PSCs injected into gastrula stage mouse embryos obtained at the post-implantation stage (Wu, J., et al. (2015). An alternative pluripotent state confers interspecies chimaeric competency. Nature 521, 316-321). The rsPSCs previously reported were generated using culture conditions that inhibited WNT signaling, using inhibitors such as IWR1. In contrast, disclosed herein is the discovery of creating naïve-like human iPSCs in a novel culture media. This novel culture media activates WNT signaling, for example by inhibiting GSK3 with CHIR99021, in combination with activators of FGF2 and Activin-A. These culture conditions result in human iPSCs with a superior capability of integrating into a blastocyst stage embryo, which was not possible with previously described cells, such as rsPSCs or conventional human PSCs. Furthermore, the present inventors integrated these naïve-like human iPSCs into a blastocyst from a mammalian species with a very different timeline of development (e.g. human vs pig or human vs cow). Such interspecies integration of cells from a species having a different developmental timeline presents additional technical challenges which have not been overcome previously in the art.

Previous approaches to answer fundamental questions in the field, including how to realize and translate the putative and theoretical advantages of the higher developmental potential associated with naïve pluripotency for human regenerative medicine as well as how to enhance our understanding of human embryogenesis and physiology, have been underdeveloped. The various culture conditions utilized towards generating naïve-like hPSCs have resulted in an inherent variability in the cells' molecular signatures, thus hampering the establishment of definitive state analogies between naïve-like hPSCs and mESCs. This has been further complicated by difficulties in developing proper in vivo functional assays able to validate human naivety. Due to ethical considerations, in vivo assays to assess the developmental potency of naïve-like hPSCs are limited to the use of animal host embryos. To date, all reported interspecies chimeric studies with naïve-like hPSCs have gravitated around the mouse as the host species. This is largely due to its accessibility, low cost of maintenance, and high reproduction rate. In addition, genetically identical mouse strains allow for more accuracy and reproducibility in often hard-to-predict chimeric studies.

Notwithstanding the importance of mouse as a premier model for stem cell research, humans and mice differ considerably in various aspects, including post-implantation epiblast development, embryo size, speed of development, and gestational period. These and other differences may affect the integration, proliferation, and differentiation of naïve-like hPSCs. Moreover, other than validating human naivety and its utility as a research model, the clinical prospects of human-mouse chimerism are limited. Large animal models provide advantages and in many cases are better predictors than mice of human clinical outcomes, and their utilization is becoming a standard prerequisite before clinical trials are performed in humans. In this regard, interspecies chimera research with large animal host(s) that is more similar to humans than mice in anatomy, physiology, and organ size, in some cases, results in both an improved research model and a more direct path towards clinical applications.

Chimeric Animals

Contemplated herein are chimeric animals, the chimeric animals comprising cells from at least two animals In some embodiments, the animals are from different species. For example, the chimeric animal may be a chimeric mammal, wherein for example, the chimeric mammal comprises cells from at least two animals from different mammalian species. The first mammal is generally the host and cells from the first mammal, at least in some cases, comprise the majority of the cells in the chimeric mammal. The second mammal is generally the donor and cells from the second mammal, at least in some cases, comprise a minority of the cells in the chimeric mammal. In some cases, the cells from the second mammal or donor mammal are induced pluripotent stem cells, such as naïve-like induced pluripotent stem cells or naïve induced pluripotent stem cells. It is further contemplated herein that the cells from the second mammal form at least one organ or tissue in the chimeric mammal, comprising the majority of the cells in that specific organ. This is accomplished, at least in part, by creation of an empty developmental niche in the first mammal which is then populated by cells from the second mammal, for example induced pluripotent stem cells (iPSCs) from the second mammal.

Chimeric animals contemplated herein, in some embodiments, comprise a first mammal with an empty developmental niche. The empty developmental niche, in some embodiments, is characterized by the absence of an organ in the first mammal. The empty developmental niche is contemplated to comprise a missing organ or tissue including liver, kidney, pancreas, hematopoietic stem cells, spleen, bone marrow, heart, lung, skin, cornea, eye, spinal cord, uterus, intestine, heart, heart valve, bone, cartilage, tendon, ligament, lymphatic vessel, central nervous system tissue, peripheral nervous system tissue, or blood vessel.

The empty developmental niche is created in the first animal by genetic modification of a zygote from the first mammal at least at one locus. A locus is generally understood herein to mean a physical location in the genome. Modifications at a locus result in inactivation or activation of one or more genes, depending on the type of modification and the precise locus that is modified. In some embodiments, a locus is modified by CRISPR/CAS methods whereby a specific modification is made at a specific site of the genome. In some embodiments, the modification includes but is not limited to deletion of all or part of the coding region of a gene, insertion of a nonsense mutation into a gene, insertion or deletion of a promoter element, insertion or deletion of an enhancer element, deletion of a splice site, or other genetic modification that, in some cases, alters expression or activity of one or more genes. In some embodiments, genetic modification includes RNAi technologies including but not limited to miRNA, siRNA, shRNA, and other gene silencing methodologies known in the art. Genetic modifications herein inactivate at least one gene, in some embodiments, including FAH, NKX2.5, TBX5, MEF2C, Osr1, Lhx1, Pax2, Pax8, Wt1, Hox11, Eya1, Six2, Sal11, Etv2, Trox1, Ronx-1, Scl/Tal-1, Lmo-2, Tel, Tek, Sox9, Scleraxis, Pax6, or Rx.

Cells of the second animal are contemplated herein to comprise induced pluripotent stem cells (iPSCs). iPSCs are known in the art as a type of pluripotent stem cell that is not derived directly from an embryo but has the capability of forming all of the tissues of a mammal iPSCs are contemplated to comprise naïve-like iPSCs, human induced pluripotent stem cells (hiPSCs), and naïve-like hiPSCs. iPSCs, in some embodiments, comprise iPSCs cultured in a culture condition including 2iLD, NHSM, 4i, or FAC.

Animals and cells from animals are used to create interspecies chimeric animals, such as chimeric mammals. The first mammal, generally understood to be a host mammal and comprising a majority of the cells in the interspecies chimeric mammal is selected on the basis of desired traits including compatibility with the second mammal or donor mammal, amenability of supporting development of cells of the second mammal into the desired organ or tissue, ease of genetic manipulation, and other traits. The chimeric mammals described herein comprise cells from a first mammal comprising mouse, rat, rabbit, guinea pig, cow, pig, horse, goat, sheep, monkey, or non-human primate. In some embodiments, the first mammal is a cow or a pig. The second mammal, generally understood to be a donor mammal and comprising a minority of the cells in the interspecies chimeric mammal, is selected on the basis of desired traits and/or desired organ to be cultured. The chimeric mammals described herein comprise cells from a second mammal comprising mouse, rat, rabbit, guinea pig, human, cow, pig, horse, goat, sheep, monkey, or non-human primate. In some embodiments, the second mammal is a human.

Chimeric animals herein are characterized by having a percentage chimerism as a ratio between the second mammal and the first mammal In some embodiments, the percentage chimerism is less than 40%. In some embodiments, the percentage chimerism is less than 30%. In some embodiments, the percentage chimerism is less than 20%. In some embodiments, the percentage chimerism is less than 10%. In some embodiments, the percentage is less than 5%. In some embodiments, the percentage chimerism is less than 1%. In some embodiments, the percentage chimerism is more than 1%. In some embodiments, the percentage chimerism is more than 5%. In some embodiments, the percentage chimerism is more than 10%. In some embodiments, the percentage chimerism is more than 20%. In some embodiments, the percentage chimerism is more than 30%. In some embodiments, the percentage chimerism is more than 40%.

Chimeric animals as contemplated herein, in some embodiments, comprise an organ or tissue derived from at least one iPSC from a second animal. The organ or tissue comprises liver, kidney, pancreas, hematopoietic stem cells, spleen, bone marrow, heart, lung, skin, cornea, eye, spinal cord, uterus, intestine, heart, heart valve, bone, cartilage, tendon, ligament, lymphatic vessel, central nervous system tissue, peripheral nervous system tissue, or blood vessel. In some embodiments, the organ or tissue in the chimeric animal is chimeric, that is, comprises cells from the second animal and the first animal. In some embodiments, the organ or tissue has a percentage chimerism as a ratio between the second animal and the first animal of at least 10%. In some embodiments, the organ or tissue has a percentage chimerism as a ratio between the second animal and the first animal of at least 50%. In some embodiments, the organ or tissue has a percentage chimerism as a ratio between the second animal and the first animal of at least 90%. In some embodiments, the organ or tissue has a percentage chimerism as a ratio between the second animal and the first animal of at least 95%. In some embodiments, the organ or tissue has a percentage chimerism as a ratio between the second animal and the first animal of at least 99%.

The chimeric animals herein, such as chimeric mammals, are contemplated, in some embodiments, to comprise a genetic modification in the first mammal that inactivates Pdx1 and comprises a cell from the second mammal which forms at least a portion of a pancreas. The chimeric mammals herein are contemplated, in some embodiments, to comprise a genetic modification in the first mammal that inactivates at least one gene including Runx-1, Scl/Tal-1, Lmo-2, Tel, or Tek and comprises a cell from the second mammal which forms at least a portion of a blood vessel or hematopoietic cell. The chimeric mammals herein are contemplated, in some embodiments, to comprise a genetic modification in the first mammal that inactivates FAH and comprises a cell from the second mammal which forms at least a portion of a liver. The chimeric mammals herein are contemplated, in some embodiments, to comprise a genetic modification in the first mammal that inactivates a gene including Osr1, Lhx1, Pax2, Pax8, Wt1, Hox11, Eya1, Six2, or Sal11 and comprises a cell from the second mammal which forms at least a portion of a kidney. The chimeric mammals herein are contemplated, in some embodiments, to comprise a genetic modification in the first mammal that inactivates a gene including Pax6 or Rx1 and comprises a cell from the second mammal which forms at least a portion of an eye. The chimeric mammals herein are contemplated, in some embodiments, to comprise a genetic modification in the first mammal that inactivates a gene including Sox9 or Scleraxis and comprises a cell from the second mammal which forms at least a portion of a cartilage tissue. The chimeric mammals herein are contemplated, in some embodiments, to comprise a genetic modification in the first mammal that inactivates a gene including Etv2 or Prox1 and comprises a cell from the second mammal which forms at least a portion of an endothelial or lymphatic vessel.

Mammalian Organs Derived from iPSCs

Contemplated herein are mammalian organs such as isolated mammalian organs derived by novel methods described herein from iPSCs, for example naïve-like iPSCs or naïve iPSCs, from a second mammal injected into a first mammal at the blastocyst stage of development. It is further contemplated that the first mammal is genetically modified to create a developmental niche for the iPSCs to form the mammalian organ. Such mammalian organs have desirable properties, such as the ability to be transplanted into a human in need of a transplant. Other properties of mammalian organs contemplated herein include the ability to test the efficacy and safety of a drug.

Isolated mammalian organs or tissues contemplated herein comprise organs or tissues cultured in a first mammal, wherein the organ or tissue is derived from an induced pluripotent stem cell (iPSC) cell from a second mammal, wherein the first and second mammal are different species. The organ or tissue, in some embodiments, comprises liver, kidney, pancreas, hematopoietic stem cells, spleen, bone marrow, heart, lung, skin, cornea, eye, spinal cord, uterus, intestine, heart, heart valve, bone, cartilage, tendon, ligament, lymphatic vessel, or blood vessel. The second mammal from which at least one iPSC is used to derive the organ or tissue comprises mouse, rat, rabbit, guinea pig, human, cow, pig, horse, goat, or sheep. In some embodiments, the second mammal is a human. A first mammal is used to culture the organ or tissue. The first mammal, in some embodiments, comprises mouse, rat, rabbit, guinea pig, cow, pig, horse, goat, or sheep. In some embodiments, the first mammal is a cow or a pig. The first mammal, in some embodiments, comprises an empty developmental niche. In some embodiments, the first mammal does not develop at least one organ or tissue. In some embodiments, the organ or tissue that is not developed comprises liver, kidney, pancreas, hematopoietic stem cells, spleen, bone marrow, heart, lung, skin, cornea, spinal cord, uterus, intestine, heart valve, bone, cartilage, tendon, ligament, or blood vessel. In some embodiments, the empty developmental niche is created by a missing organ or tissue in the first mammal. The first mammal, in some embodiments, comprises a genetic modification at one or more loci. In some embodiments, the genetic modification inactivates at least one gene including FAH, NKX2.5, TBX5, MEF2C, Osr1, Lhx1, Pax2, Pax8, Wt1, Hox11, Eya1, Six2, Sal11, Etv2, Trox1, Ronx-1, Scl/Tal-1, Lmo-2, Tel, Tek, Sox9, Scleraxis, Pax6, or Rx.

Isolated mammalian organs contemplated herein are derived from an iPSC from a second mammal. iPSCs are known in the art as a type of pluripotent stem cell that is not derived directly from an embryo but has the capability of forming all of the tissues of a mammal. iPSCs are contemplated to comprise naïve iPSCs, naïve-like iPSCs, hiPSCs, and naïve-like hiPSCs. iPSCs, in some embodiments, comprise iPSCs cultured in a culture condition including 2iLD, NHSM, 4i, or FAC.

The isolated mammalian organs or tissues herein are contemplated, in some embodiments, to be cultured in a first mammal comprising a genetic modification that inactivates Pdx1 and the cell from the second mammal which forms at least a portion of a pancreas. The isolated mammalian organs or tissues herein are contemplated, in some embodiments, to be cultured in a first mammal comprising a genetic modification that inactivates at least one gene including Runx-1, Scl/Tal-1, Lmo-2, Tel, or Tek and the cell from the second mammal which forms at least a portion of a blood vessel or hematopoietic cell. The isolated mammalian organs or tissues herein are contemplated, in some embodiments, to be cultured in a first mammal comprising a genetic modification that inactivates FAH and the cell from the second mammal which forms at least a portion of a liver. The isolated mammalian organs or tissues herein are contemplated, in some embodiments, to be cultured in a first mammal comprising a genetic modification that inactivates a gene including Osr1, Lhx1, Pax2, Pax8, Wt1, Hox11, Eya1, Six2, or Sal11 and the cell from the second mammal which forms at least a portion of a kidney. The isolated mammalian organs or tissues herein are contemplated, in some embodiments, to be cultured in a first mammal comprising a genetic modification that inactivates a gene including Pax6 or Rx1 and the cell from the second mammal which forms at least a portion of an eye. The isolated mammalian organs or tissues herein are contemplated, in some embodiments, to be cultured in a first mammal comprising a genetic modification that inactivates a gene including Sox9 or Scleraxis and the cell from the second mammal which forms at least a portion of a cartilage tissue. The isolated mammalian organs or tissues herein are contemplated, in some embodiments, to be cultured in a first mammal comprising a genetic modification that inactivates a gene including Etv2 or Prox1 and the cell from the second mammal which forms at least a portion of an endothelial or lymphatic vessel.

Isolated mammalian organs and tissues contemplated herein are stored prior to use in a physiologically acceptable buffer or excipient. In some embodiments, the isolated mammalian organ or tissue is stored prior to use in a buffer or excipient that preserves the organ or tissue so that it maintains viability of the organ or tissue for transplantation into a patient. In some embodiments, the isolated organ or tissue is stored at approximately 4° C. In some embodiments, the isolated organ or tissue is stored at approximately 25° C. In some embodiments, the isolated organ or tissue is stored at approximately 37° C. In some embodiments, the isolated organ or tissue is stored at approximately 0° C.

Methods of Culturing Mammalian Organs

Contemplated herein are methods of culturing a mammalian organ or tissue, for example a cultured human organ or tissue that is useful in the treatment of a subject, for example a human subject, in need of treatment, for example an organ or tissue transplant. Some such methods of culturing a mammalian organ or tissue comprise injecting a blastocyst of a first mammal with at least one iPSC, for example a naïve-like iPSC, from a second mammal to form a chimeric blastocyst; transferring the chimeric blastocyst into a pseudo-pregnant third mammal; and obtaining a chimeric mammal comprising the mammalian organ or tissue; wherein the first and second mammal are different species, and wherein the first mammal is genetically modified at one or more loci and the organ or tissue is derived from iPSC from the second mammal.

Organs and tissues contemplated for culture include liver, kidney, pancreas, hematopoietic stem cells, spleen, bone marrow, heart, lung, skin, cornea, eye, spinal cord, uterus, intestine, heart valve, bone, cartilage, tendon, ligament, central nervous system tissue, peripheral nervous system tissue, lymphatic vessel, or blood vessel.

iPSCs are contemplated to be derived from a second mammal In some embodiments, the second mammal comprises mouse, rat, rabbit, guinea pig, human, cow, pig, horse, goat, or sheep. In some embodiments, the second mammal is a human. In some embodiments, the iPSC is derived from a hiPSC type comprising an hiPSC cultured in a culture condition including 2iLD, NHSM, 4i, or FAC. In some embodiments, the iPSC is a naïve-like iPSC or naïve-like hiPSC.

Methods of culturing mammalian organs or tissues comprise use of a first mammal, for example a host mammal, from which a blastocyst is derived for injection of iPSCs from the second mammal. Methods of culturing mammalian organs or tissues also comprise the use of a third mammal, for example a pseudo-pregnant female, into which the chimeric blastocyst is transferred or implanted. In some embodiments, the first mammal and the third mammal comprise mouse, rat, rabbit, guinea pig, cow, pig, horse, goat, or sheep. In some embodiments, the first mammal and the third mammal are a cow or a pig. In some embodiments, the first mammal and the third mammal are the same species. In some embodiments, the blastocyst of the first mammal is genetically modified at a locus by a method comprising contacting a zygote of the first mammal with a CRISPR/CAS reagent that inactivates at least one gene at the locus. In some embodiments, the first mammal is genetically modified at a locus that inactivates at least one gene including FAH, NKX2.5, TBX5, MEF2C, Osr1, Lhx1, Pax2, Pax8, Wt1, Hox11, Eya1, Six2, Sal11, Etv2, Trox1, Ronx-1, Scl/Tal-1, Lmo-2, Tel, Tek, Sox9, Scleraxis, Pax6, or Rx.

Methods of culturing a mammalian organ or tissue comprise creation of a chimeric blastocyst. Chimeric blastocysts are created by methods known in the art, for example, injection of an iPSC, for example 1, 2, 3, 4, 5, 6, 7, 8, 9, 10, 11, 12, 13, 14, 15, 16, 17, 18, 19, or 20 iPSCs from the second mammal are injected into the blastocyst of the first mammal. In some embodiments, 1-5 iPSCs from the second mammal are injected into the blastocyst of the first mammal. In some embodiments, 1-10 iPSCs from the second mammal are injected into the blastocyst of the first mammal. In some embodiments, 1-15 iPSCs from the second mammal are injected into the blastocyst of the first mammal. In some embodiments, 1-5, 1-10, 1-15, 1-20, 5-10, 5-15, or 5-20 iPSCs from the second mammal are injected into the blastocyst of the first mammal In some embodiments, the blastocyst of the first mammal is injected between days 4 and 7 post-fertilization, for example on day 4, 5, 6, or 7 post-fertilization. In some embodiments, the blastocyst of the first mammal is injected between days 3 and 8, days 4 and 7, days 5 and 6, days 4 and 8, or days 3 and 7 post-fertilization. In some embodiments, the chimeric blastocyst is transferred 2 to 30 hours after injection, for example 2, 3, 4, 5, 6, 7, 8, 9, 10, 11, 12, 13, 14, 15, 16, 17, 18, 19, 20, 21, 22, 23, 24, 25, 26, 27, 28 ,29, or 30 hours after injection. In some embodiments, the chimeric blastocyst is transferred 2 to 5, 5 to 10, 10 to 15, 15 to 20, 20 to 25, or 25 to 30 hours after injection.

In some embodiments, the first mammal comprises a genetic modification that inactivates Pdx1 and the cells from the second mammal form at least a portion of a pancreas. In some embodiments, the first mammal comprises genetic modifications that inactivate one or more genes including Runx-1, Scl/Tal-1, Lmo-2, Tel, and/or Tek and the cells from the second mammal form at least a portion of a blood vessel. In some embodiments, the first mammal comprises genetic modifications that inactivate FAH and the cells from the second mammal form at least a portion of a liver. In some embodiments, the first mammal comprises genetic modifications that inactivate one or more genes including Osr1, Lhx1, Pax2, Pax8, Wt1, Hox11, Eya1, Six2, and/or Sal11 and the cells from the second mammal form at least a portion of a kidney. In some embodiments, the first mammal comprises genetic modifications that inactivate one or more genes including Nkx2.5, Tbx5, and/or Mef2c and the cells from the second mammal form at least a portion of a heart. In some embodiments, the first mammal comprises genetic modifications that inactivate one or more genes including Pax6 and/or Rx1 and the cells from the second mammal form at least a portion of an eye. In some embodiments, the first mammal comprises genetic modifications that inactivate one or more genes including Sox9 and/or Scleraxis and the cells from the second mammal form at least a portion of a cartilage tissue. In some embodiments, the first mammal comprises genetic modifications that inactivate one or more genes including Etv2 and/or Prox1 and the cells from the second mammal form at least a portion of an endothelial or lymphatic vessel.

Methods of Treatment

Contemplated herein are methods of treating an individual in need of an organ or a tissue transplant using cultured mammalian organs described herein. Cultured mammalian organs herein have the advantage that they are derived from an iPSC, such as a human iPSC, and they are cultured or grown in a chimeric mammal. Therefore, cultured mammalian organs are customizable and are not dependent on organ donor volunteers. Additional advantages of mammalian organs herein are suitability for treatment of each individual as the iPSC, from which the organ or tissue is derived, is obtainable from the individual in need of organ or tissue transplant and the organ or tissue is HLA matched to the individual receiving the transplant.

Methods of treating an individual in need of an organ or a tissue transplant comprise administration or transplant of a tissue or organ described herein. Organs for transplant are contemplated to include but are not limited to liver, kidney, pancreas, hematopoietic stem cells, spleen, bone marrow, heart, lung, skin, cornea, eye, spinal cord, uterus, intestine, heart valve, bone, cartilage, tendon, ligament, central nervous system tissue, peripheral nervous system tissue, lymphatic vessel, or blood vessel.

Individuals in need of transplant include but are not limited to individuals diagnosed with or suffering from a disease including but not limited to liver failure, liver cirrhosis, kidney failure, diabetes, cancer, severe combined immunodeficiency syndrome, aplastic anemia, congenital neutropenia, sickle cell anemia, thalassemia, intestine failure, ruptured spleen, heart failure, coronary artery disease, congenital heart disease, burn, skin infection, skin cancer, venous ulcer, pressure ulcer, diabetic ulcer, wound, macular degeneration, glaucoma, retinoblastoma, complete paraplegia, complete tetraplegia, anterior cord syndrome, central cord syndrome, posterior cord syndrome, Brown-Sequard syndrome, Cauda Equina Lesion, congenital uterine disease, female infertility, intestinal failure, parenteral nutrition disorder, chronic obstructive pulmonary disease, emphysema, idiopathic pulmonary fibrosis, cystic fibrosis, idiopathic pulmonary hypertension, alpha 1-antitrypsin deficiency, bronchiectasis, sarcoidosis, keraconus, Fuchs' dystrophy, cornea thinning, cornea scarring, cornea clouding, cornea swelling, corneal ulcer, bone cancer, bone fracture, rheumatoid arthritis, osteoarthritis, ruptured tendon, amyotrophic lateral sclerosis, Parkinson's disease, Alzheimer's disease, Huntington's disease, aging, dementia, neurodegeneration, multiple sclerosis, congenital vascular disorder, and atherosclerosis.

Organs derived from iPSCs for treatment of individuals are derived from the same individual in need of a transplant. iPSCs are also contemplated to be derived from a first-degree relative. It is also contemplated that iPSCs are derived from an unrelated HLA-matched individual. An HLA-matched individual is understood to be an individual who matches HLA-type with the individual in need of a transplant at a minimum of five of five loci. It is also understood that an HLA-matched individual matches HLA-type with the individual in need of a transplant at a minimum of ten out of ten loci. In some embodiments, an HLA-matched individual is a partial match, matching the HLA-type with the individual in need of a transplant at four out of five, eight out of ten, or nine out of ten loci.

Methods of Drug Safety and Efficacy Testing

Contemplated herein are novel methods of testing safety and efficacy of a drug, for example using a chimeric mammal described herein to more accurately determine the safety and efficacy of a therapeutic drug prior to testing in human subjects. Limitations in current pre-clinical safety and efficacy testing of a drug are that the target organ or tissue for the drug in the test organism is a different species than the intended target organ or tissue. For example, testing a drug that targets the kidney for therapy can currently be tested in a model organism, but differences in the kidney of a model organism compared to a human subject can lead to inaccurate results. Therefore, it is desirable to test a drug in a more relevant model organism, such as a chimeric mammal described herein, where the target organ or tissue is derived from a human and more accurate results will be achieved in such testing. Some such methods of testing safety and efficacy include a method of testing the safety and efficacy of a drug comprising administering the drug to any of the chimeric mammals described herein and measuring toxicity and therapeutic benefit in the chimeric mammal In some embodiments, the toxicity comprises at least one of body weight, food consumption, histopathology, immunogenicity, autoimmunity, genotoxicity, nephropathy, hepatopathology, neuropathology, and carcinogenesis. In some embodiments, the therapeutic benefit comprises an intended effect of the drug administered.

Also contemplated herein are in vitro methods of testing safety and efficacy. In some embodiments, the method of testing the safety and efficacy of a drug comprises contacting the drug to any of the mammalian organs described herein and measuring toxicity and in vitro therapeutic benefit. In some embodiments, the toxicity comprises at least one of cellular apoptosis, histopathology, and genotoxicity. In some embodiments, the in vitro therapeutic benefit comprises an intended effect of the drug administered.

Naïve-like Induced Pluripotent Stem Cells

Also contemplated herein are methods of culturing induced pluripotent stem cells, such as naïve induced pluripotent stem cells. As used herein, “naïve-like induced pluripotent stem cells” or “naïve-like iPSCs”, used interchangeably herein, are induced pluripotent stem cells in a naïve state that have the capability of differentiating into any cell or tissue in the body and are capable of being integrated into an embryo at the blastocyst stage. In some embodiments, naïve-like iPSCs resemble early epiblast cells in pre-implantation embryos. In some embodiments, naïve-like iPSCs show global DNA hypomethylation. In some embodiments, female naïve-like iPSCs have two active X chromosomes. In some embodiments, naïve-like iPSCs are positive for markers including Klf4 and Tfcp211. In some embodiments, human naïve-like iPSCs are positive for markers including KLF17. In some embodiments, mouse naïve-like iPSCs or naïve iPSCs are positive for markers including Klf2 and Esrrb. In some embodiments, naïve-like iPSCs are able to generate chimeras following injection into a blastocyst. Some such methods include a method of making an induced pluripotent stem cell (iPSC) comprising: transfecting a population of human foreskin fibroblasts (HFF) with at least three episomal vectors comprising pCXLE-hOCT3/4-shp53-F, pCXLE-hSK, and pCXLE-hUL; contacting the transfected population of HFFs with a population of mitotically inactivated mouse embryonic fibroblasts (MEFs); contacting the population of HFFs and the population of MEFs with an FAC medium comprising a base media (for example comprising DMEM/F12, Neurobasal medium, N2 supplement, B27 supplement, GlutaMax, non-essential amino acids, beta-mercaptoethanol, antibiotics) with the additional factors such as an FGF, an Activin, and a GSK inhibitor until at least one colony comprising an iPSC has formed; and transferring the colony comprising an iPSC to a fresh population of mitotically inactivated MEFs. In some embodiments, the FGF comprises FGF2, FGF9, FGF20, FGF1, FGF4, FGF19, or FGF21. In some embodiments, the additional factors comprise a WNT pathway growth factor, for example Wnt3, Wnt-3a, Wnt7b, or Wnt-5a. In some embodiments, the additional factors include FGF2, Activin-A, and CHIR99021. In some embodiments, the HFF are also transfected with the episomal vector, pCXLE-EGFP. In some embodiments, the FAC medium comprises bovine serum albumin.

Such naïve-like induced pluripotent stem cells created using methods herein are useful in treating disease, including in methods of making chimeric animals and organs from chimeric animals, such as those disclosed herein.

### EXAMPLES

The following examples are given for the purpose of illustrating various embodiments of the invention and are not meant to limit the present invention in any fashion. The present examples, along with the methods described herein are presently representative of embodiments, are exemplary, and are not intended as limitations on the scope of the invention. Changes therein and other uses which are encompassed within the spirit of the invention as defined by the scope of the claims will occur to those skilled in the art.

### Example 1

### Generation of Naïve-like hiPSCs

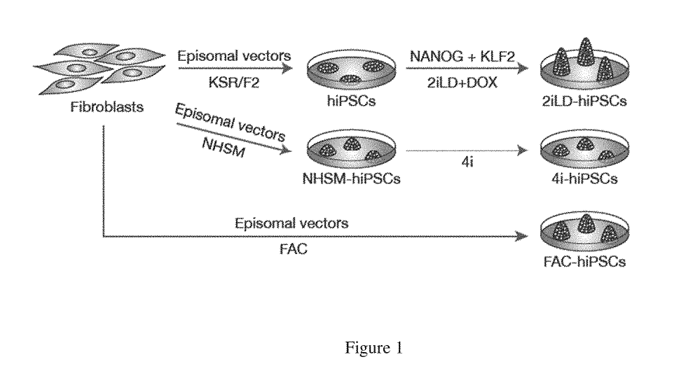

With the goal of empirically testing chimeric contribution of naïve-like hiPSCs in animal hosts that bear more physiological resemblance to humans, four different types of naïve-like hiPSCs were generated either through conversion from primed hiPSC line or direct induction under naïve culture conditions (FIG. 1).

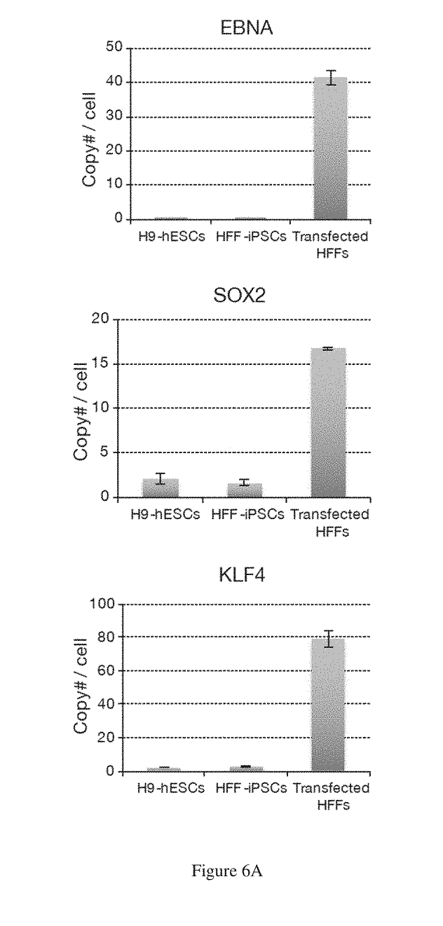

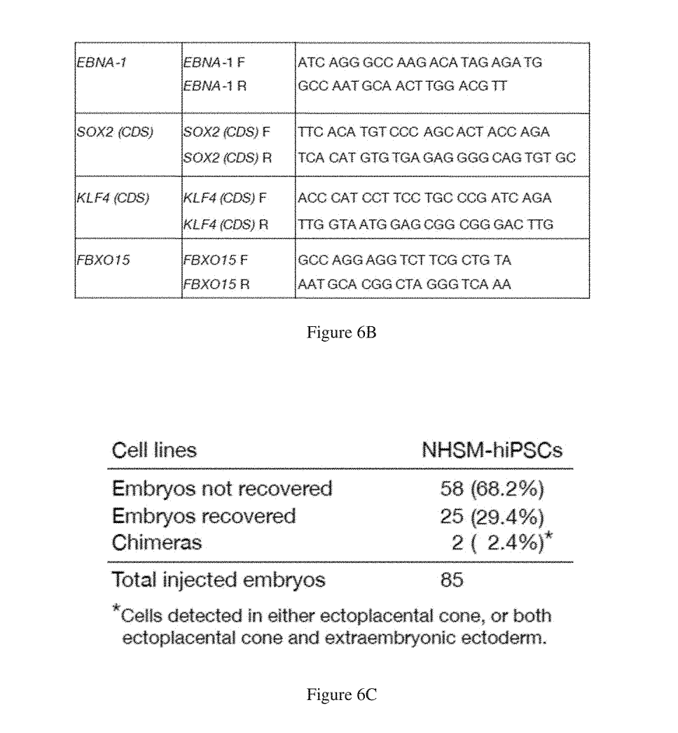

Mouse ground state culture condition (2iL) induces differentiation of conventional hPSCs. However, when combined with over-expression of NANOG and KLF2 (NK2), two key transcription factors in maintaining murine naïve pluripotency, 2iL culture is able to stabilize hPSCs in a naïve-like state. These NK2-overexpressing cells have been used in several studies as starting populations to screen for small molecules that can elicit self-renewal of a naïve-like program in hPSCs independent of transgene expression. For this example, transgene-dependent naïve-like hiPSCs were generated from primed hiPSCs (FIG. 1). Primed hiPSCs were obtained by reprogramming human foreskin fibroblasts (HFFs) using episomal vectors and cultured in conventional KSR/F2 medium on mitotically inactivated mouse embryonic fibroblasts (MEFs). The primed hiPSCs were confirmed to be devoid of exogenous reprogramming factors expression and maintained a normal diploid karyotype (FIG. 6A, FIG. 6B, FIG. 6C). Next, primed hiPSCs were infected with lentiviruses encoding rtTA and a doxycycline (DOX)-inducible NK2 followed by selection of drug-resistant clones in 2iL culture with the supplementation of DOX. Consistent with previous reports, NK2-overexpression via DOX treatment combined with 2iL culture (2iLD) was able to allow for the generation and maintenance of naïve-like hiPSCs with dome shaped morphology. For comparison, naïve-like hiPSCs were also generated from HFFs using a modified NHSM culture system (NHSM-hiPSCs, FIG. 1). NHSM-hiPSCs could be stably maintained in the NHSM condition with a colony morphology that was flatter than that of 2iLD-hiPSCs. Naive-like hPSCs cultured in NHSM have been tested for their developmental potency in vivo using mouse hosts but with contrasting results. The chimeric contribution of NHSM-hiPSCs was also tested using mouse as the host. Interestingly, although robust and consistent integration was observed of NHSM-hiPSCs into the mouse inner cell mass (ICM) after blastocyst injection, upon embryo transfer to pseudopregnant surrogates, contribution of Kusabira Orange (KO)-labeled NHSM-hiPSCs to the embryo proper was not detected, but instead, observed limited contribution of NHSM-hiPSCs to extraembryonic lineages. It has been shown that cells grown in 4i medium, a simplified version of NHSM, have a significant potential for germ cell induction, a distinguishing feature of naïve mESCs and primed EpiSCs. In this regard, NHSM-hiPSCs were culture-adapted in 4i medium (4i-hiPSCs, FIG. 1), resulting in stable 4i-hiPSCs with similar morphological characteristics to parental NHSM-hiPSCs. 2iLD-hiPSCs, NHSM-hiPSCs, and 4i-hiPSCs all could be stably maintained after long term culture and expressed OCT4 protein in their nucleus.

In addition, a new type of hiPSC was generated by direct reprogramming of HFFs in a modified mouse EpiSCs medium (FAC, FIG. 1). Mouse EpiSCs cultured in FAC medium were able to generate germline competent chimeras, a feature normally associated with naïve mESCs. hiPSCs generated and cultured in FAC medium (FAC-hiPSCs) displayed a colony morphology intermediate between that of 2iLD cells and NHSM cells with less defined borders. FAC-hiPSCs could be efficiently propagated by single-cell dissociation and maintained a normal karyotype after long-term culture. Immunofluorescence analysis showed expression of pluripotent markers OCT4, NANOG, TRA-1-60, and TRA-1-80 in FAC-hiPSCs.

After injection into kidney capsules of immunodeficient mice, all naïve-like hiPSCs formed teratomas consisting of cells from all three germ layers: Endoderm, Mesoderm, and Ectoderm. To facilitate identification of human derived cells in subsequent chimeric experiments, the naïve-like hPSCs were labeled with either green fluorescence protein (GFP) (2iLD-hiPSCs and FAC-hiPSCS) or KO fluorescent markers (NHSM-hiPSCs and 4i-hiPSCs).

### Example 2

### Interspecies Human-Cattle Chimeric ICM Formation with Naïve-like hiPSCs

Studies with naïve and primed PSCs, two major types of stem cells from early embryos, suggested that matching the spatiotemporal properties of PSCs to the different developmental stages of the host embryo is key for their efficient in vivo engraftment and generation of intra- and inter-species chimeras. Naive mESCs and primed EpiSCs were derived from and bear molecular properties similar to the pre-implantation epiblast and post-implantation epiblast respectively. Thus, after being grafted back to a developing embryo, mESCs thrive in pre-implantation blastocysts but not in the post-implantation embryo. On the other hand, EpiSCs cannot efficiently integrate into the ICM while they robustly integrate, proliferate, and differentiate in gastrula stage epiblasts. In line with this observation, interspecies human-mouse chimeric studies also demonstrated efficient engraftment of primed hPSCs to post-implantation gastrula stage embryos ex vivo; and incorporation of naïve-like hPSCs to mouse ICMs after blastocyst transfer. Thus, the ICM incorporation assay can be informative for evaluating whether naïve-like hiPSCs are compatible with the pre-implantation epiblasts of large animals, the first important step towards the generation of chimeric embryos. To this end, the ability of naïve-like hiPSCs to form interspecies chimeric ICM in cattle was first evaluated.

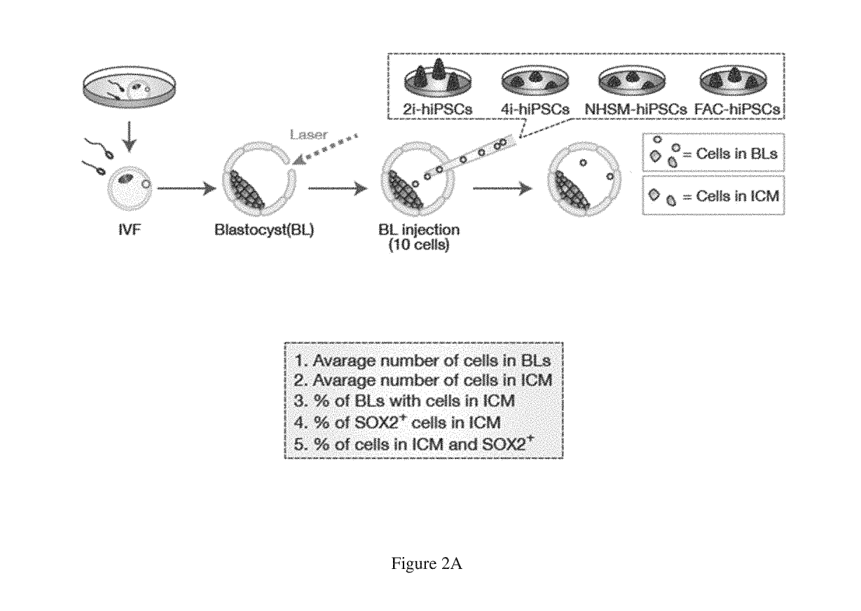

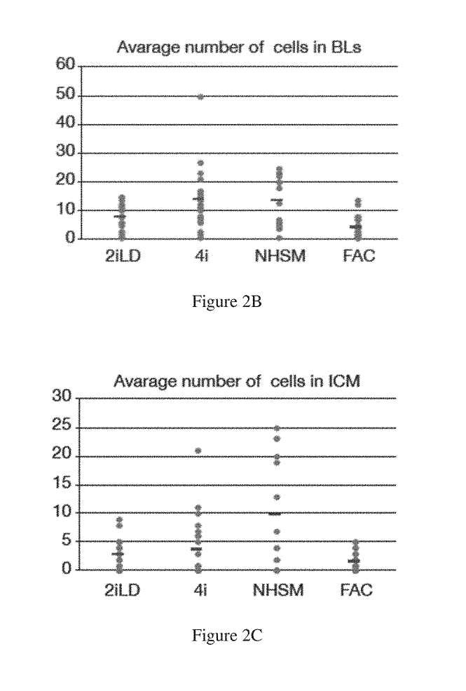

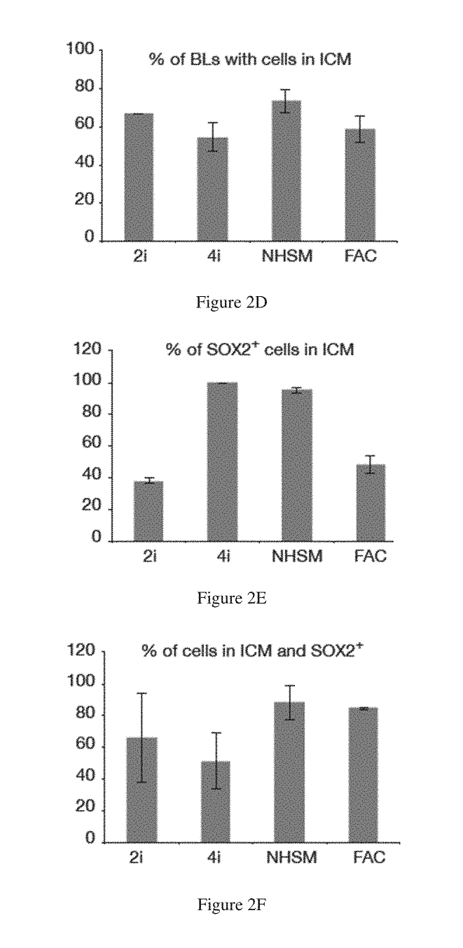

Cattle assisted reproductive technologies, such as in vitro embryo production, are well developed given its commercial importance for improved animal genetics and also as a research model with several similar characteristics to human pre-implantation development. Based on in vitro produced bovine embryos, a system was developed to test for the ability and efficiency of naïve-like hiPSCs to survive in the blastocyst environment and integrate into the bovine ICM, a potential functional test for naivety (FIG. 2A). Bovine embryos were obtained by in vitro fertilization (IVF) using in vitro matured oocytes collected from slaughterhouse-derived ovaries.

The tightly connected cells of the blastocyst trophectoderm from large livestock species such as pig and cattle form a barrier that complicates cell microinjection into the blastocoel (blastocyst cavity), with attempts often resulting in embryo collapse and inability to deposit the cells into the embryo. To facilitate cell injection, a laser-assisted approach was employed, based on using a laser beam to perforate the zona pellucida and induce damage to a limited number of trophectoderm cells, allowing for easy access into the blastocyst cavity for transferring the human cells (FIG. 2A). Furthermore, the zona ablation and trophectoderm access allowed for using a blunt end pipette for cell transfer, thus minimizing further embryo damage. This method resulted in almost 100% injection effectiveness and >90% embryo survival.

To determine the potential of naïve-like hPSCs for engrafting into the bovine blastocysts 10 cells from different types of naïve-like conditions were injected into cattle blastocysts collected seven days after fertilization and their chimeric contribution to the ICM was evaluated. After hiPSC cell injection, blastocysts were first cultured in naïve hiPSC medium for 4 hours and then changed to a mixed medium (naïve hiPSC medium and KSOM embryo culture medium mixed at 1:1 ratio) for 20 hours. Subsequently, the blastocysts were transferred to bovine embryo culture medium (KSOM) and cultured for an additional 24 hours before fixation for immunostaining with anti-human and anti-SOX2 antibodies. Several criteria were used to evaluate the chimeric contribution of naïve-like hiPSCs to cattle blastocysts which included: 1) average number of human cells in each blastocyst; 2) average number of human cells in the ICM; 3) percentage of blastocysts with human cells residing in the ICM; 4) percentage of SOX2+ human cells that reside in the ICM; 5) percentage of human cells residing in the ICM that are SOX2+ (FIG. 2A-F and Table 1). These results indicated that although all four types of naïve-like hiPSCs could survive and integrate into bovine ICMs, they displayed variable efficiency (FIG. 2D, 2F). Compared to other cell types, 4i-hiPSCs survived the best in bovine blastocysts (22/23 blastocysts contained human cells) but the majority lost SOX2 expression (only 13.6% of human cells remained SOX2+) (Table 1). On average 3.64 4i-hiPSCs were incorporated into the ICM (FIG. 2C). NHSM-hiPSCs were detected in 15/26 of blastocysts after 2 days of embryo culture with an average of 9.47 cells showing ICM localization, of which 95.4% remained SOX2+ (FIG. 2C and Table 1). 2iLD-hiPSCs and FAC-hiPSCs exhibited moderate but consistent survival rates and ICM incorporation efficiency, with an average 2.67 and 1.32 cells per ICM respectively (FIG. 2C). Interestingly, we observed a fraction of human cells staining positive for SOX2 but residing outside the ICM, and this population varied between cell types used (2iLD, 62.2%; 4i, 0%, NHSM, 4.6% and FAC, 51.9%) (FIG. 2E and Table 1). In sum, the efficacy of using in vitro produced bovine embryos for evaluating naïve-like hiPSCs' contribution to pre-implantation bovine epiblasts was demonstrated, the first indicator of their chimeric competency. Also, laser-assisted microinjection proves to be an effective way of delivering donor cells into the blastocyst cavity. Unlike primed hiPSCs, which showed little to no ICM incorporation, it was observed that consistent survival and ICM incorporation of all naïve-like hiPSC types used in this study. It is important to note that the capacity of naïve-like hiPSCs to survive and engraft in the bovine ICM varied among the different naïve culture conditions used.

### Example 3

### Interspecies Human-Pig Chimeric ICM Formation with Naïve-like hiPSCs

The pig has certain advantages over cattle for experiments involving post-implantation embryos, such as being a polytocus species and a more commonly used translational model given its similarities to humans in organ physiology, size, and anatomy.

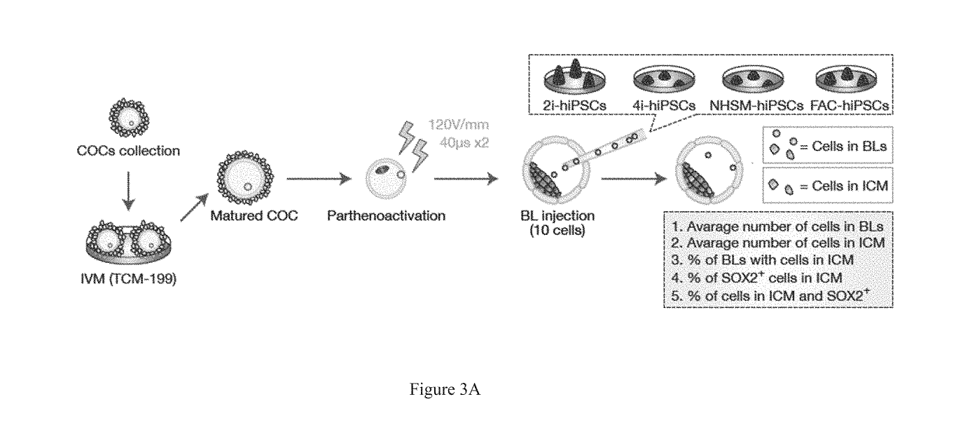

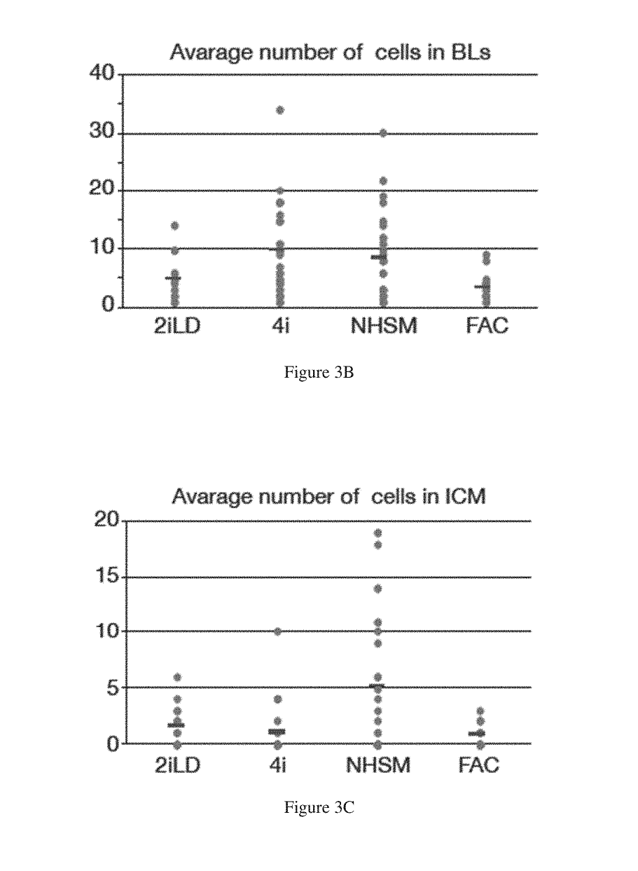

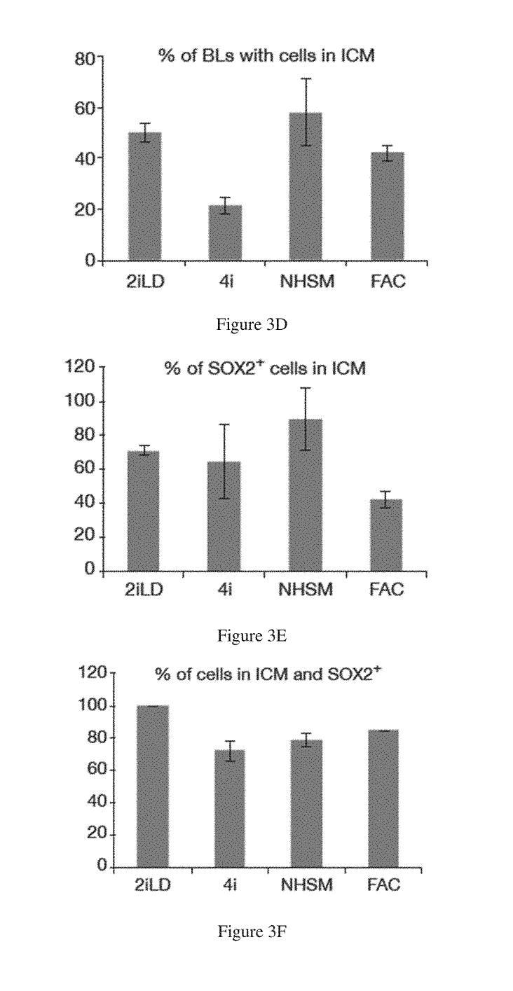

Due to the common complications with pig IVF, such as high levels of polyspermic fertilization, a parthenogenetic activation model was used allowing for efficient production of embryos that develop to blastocysts and able to generate post-implantation embryos. Embryos obtained through this methodology were not able to develop to full term because of later fetal abnormalities associated with aberrant imprinting. Pig oocytes were obtained from ovaries collected at a local slaughterhouse and matured in vitro for 44-48 hrs. Subsequently, after removing the cumulus cells, the oocytes were artificially activated using electrical stimulation. Activated oocytes were cultured in PZM5 medium for 5-6 days at which point blastocysts were selected and injected with human pluripotent cells as described for bovine blastocysts (FIG. 3A).

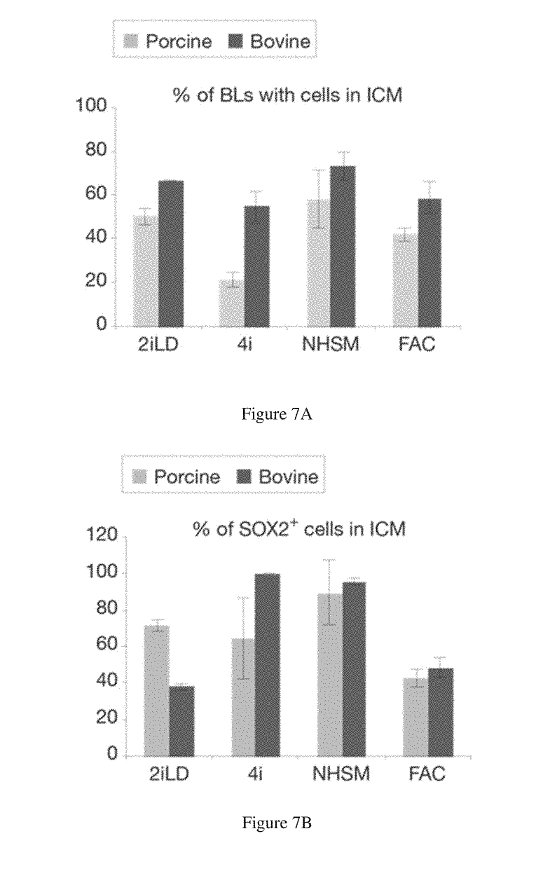

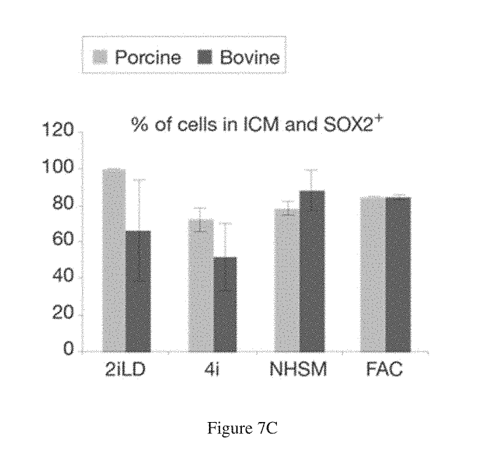

Similar to the cattle experiments, 10 cells were injected to each pig parthenogenetic blastocyst and their chimeric contribution was evaluated after successive culture in hiPSC medium for 4 hours, mixed medium for 20 hours, and porcine embryo culture medium (PZM5) for 24 hours. The chimeric contribution of naïve-like hiPSCs to pig blastocysts was evaluated after immunostaining with anti-human and anti-SOX2 antibodies using the same criteria as described for bovine blastocysts (FIG. 3A-3G and Table 1). It was found that naïve-like hiPSCs cultured in 4i and NHSM media survived better and yielded a higher percentage of blastocysts (80.0% and 81.6% respectively) harboring human cells after 48 hours of in vitro embryo culture (Table 1). Also, among all blastocysts containing human cells, on average 9.5 cells per blastocyst were observed for 4i-hiPSCs and 8.13 for NHSM-hiPSCs (FIG. 3B). For NHSM-hiPSCs 58.1% of blastocysts had human cells located in the ICM. In contrast, only 17.9% of blastocysts with ICM localization of 4i-hiPSCs were observed (Table 1). For 2iLD-hiPSCs, we observed 61.5% of blastocysts with an average of 4.81 cells per blastocyst and 50.5% of blastocysts with human cells localized to the ICM (FIG. 3C and Table 1). For FAC-hiPSCs, an average of 3.42 cells per blastocyst were present in 70.4% of blastocysts injected but only 36.8% of them had human cells with ICM integration (FIG. 3C, 3E and Table 1). Once within the ICM, a majority of the cells stained positive for the pluripotent marker SOX2 regardless of culture conditions (FIG. 3F). Similar to the observation in cattle, SOX2+ cells were observed located outside of porcine ICMs (2iLD, 29%; 4i, 35.7%, NHSM, 10.9% and FAC, 57.7%) (FIG. 3E and Table 1). Moreover, the percentage of SOX2+ cells also varied between cell types (Table 1). When comparing the results of cattle and pig, it was noticed that naïve-like hiPSCs seemed to perform better in bovine blastocysts than porcine ones (FIG. 7A, FIG. 7B, FIG. 7C and Table 1), suggesting that the different in vivo blastocyst environment between pig and cattle and likely bovine blastocysts are more permissive for naïve-like hiPSCs.

Taken together, this method of using porcine blastocysts produced via parthenogenesis as hosts bypasses some of the complications associated with pig IVF and serves as a robust platform for examining chimeric contribution of naïve-like hiPSCs. Similar to what was observed in cattle, chimeric ICM formation using porcine blastocysts indicated that survival, propensity for differentiation, and degree and efficiency of ICM incorporation vary between the different naïve-like hiPSCs examined. Thus, these observations suggest that distinct in vitro culture systems may endow different epigenetic features that ultimately affect the fate of donor human cells within the pre-implantation blastocyst environment. Consistent with the bovine results, and notwithstanding the variable efficiency and degree of ICM incorporation, it was found that all naïve-like hiPSCs examined in this study were capable of engrafting to the ICM of porcine embryos while expressing the pluripotency marker SOX2.

### Example 4

### Effects of Injection Timing on Interspecies Chimeric ICM Formation

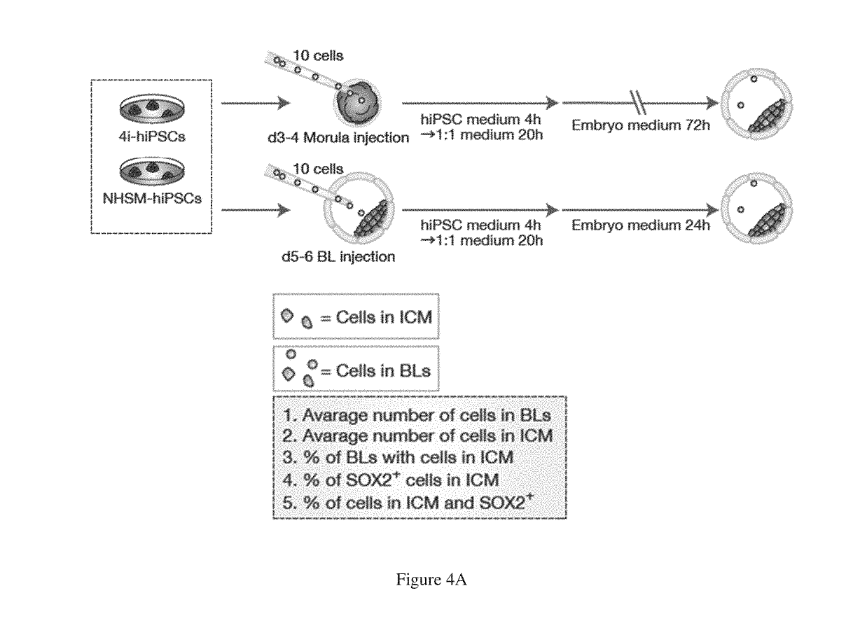

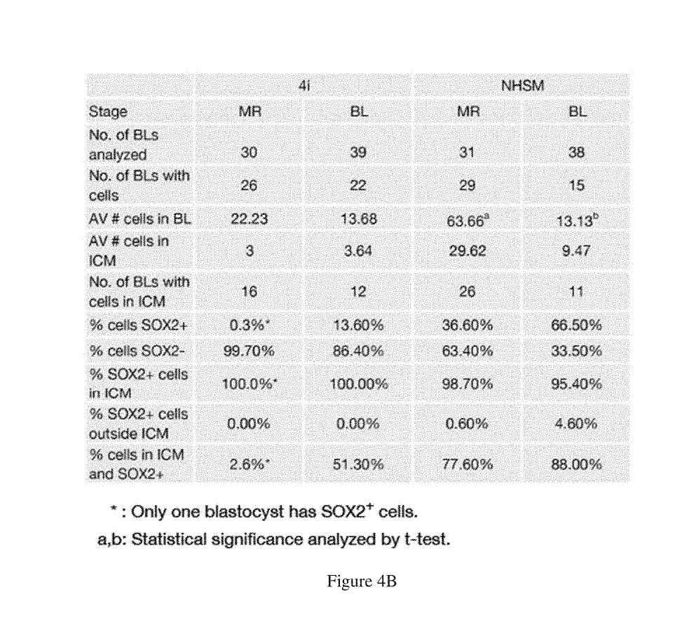

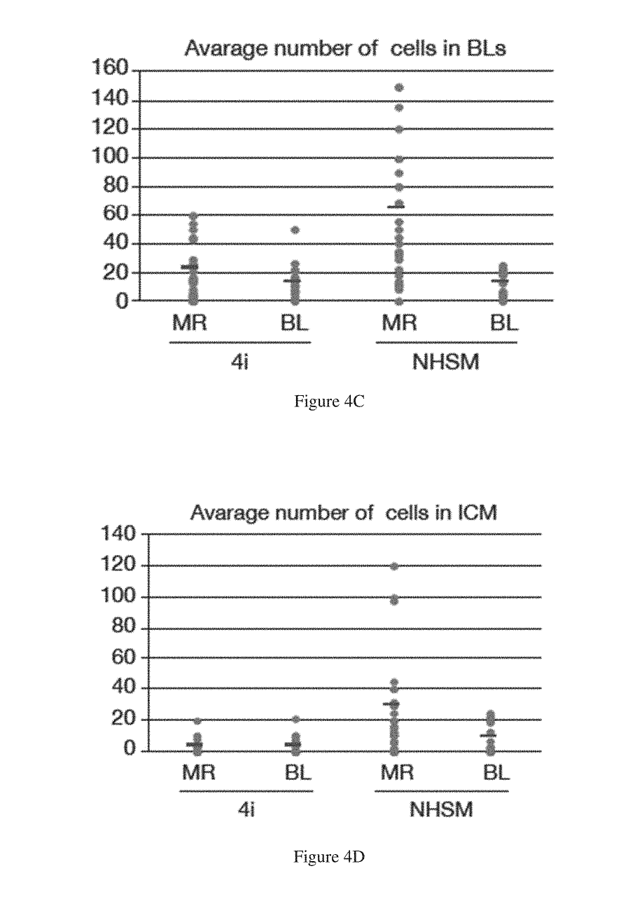

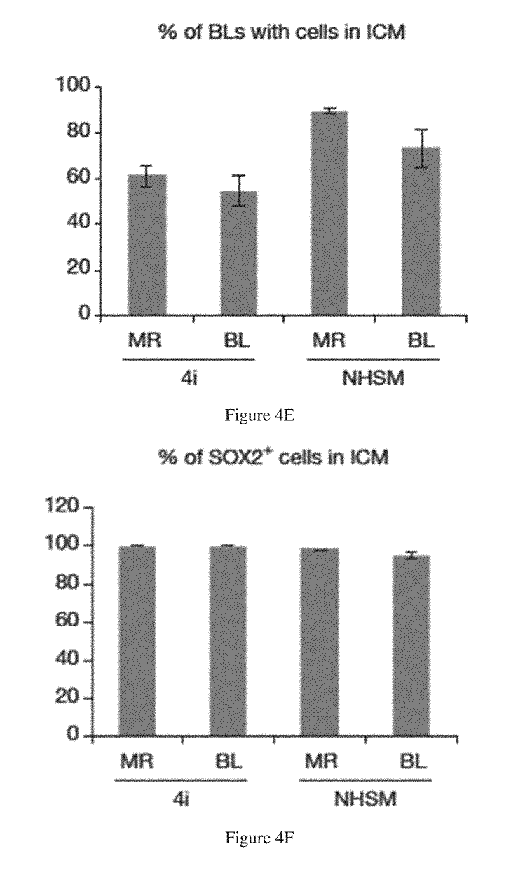

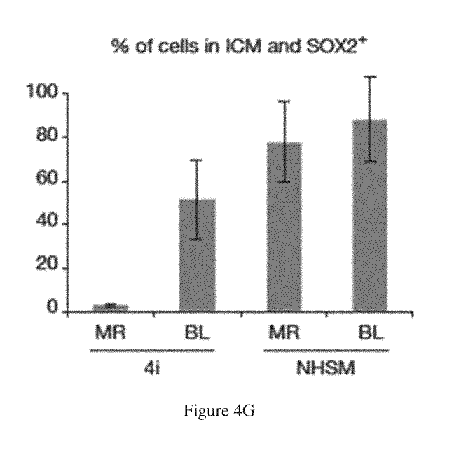

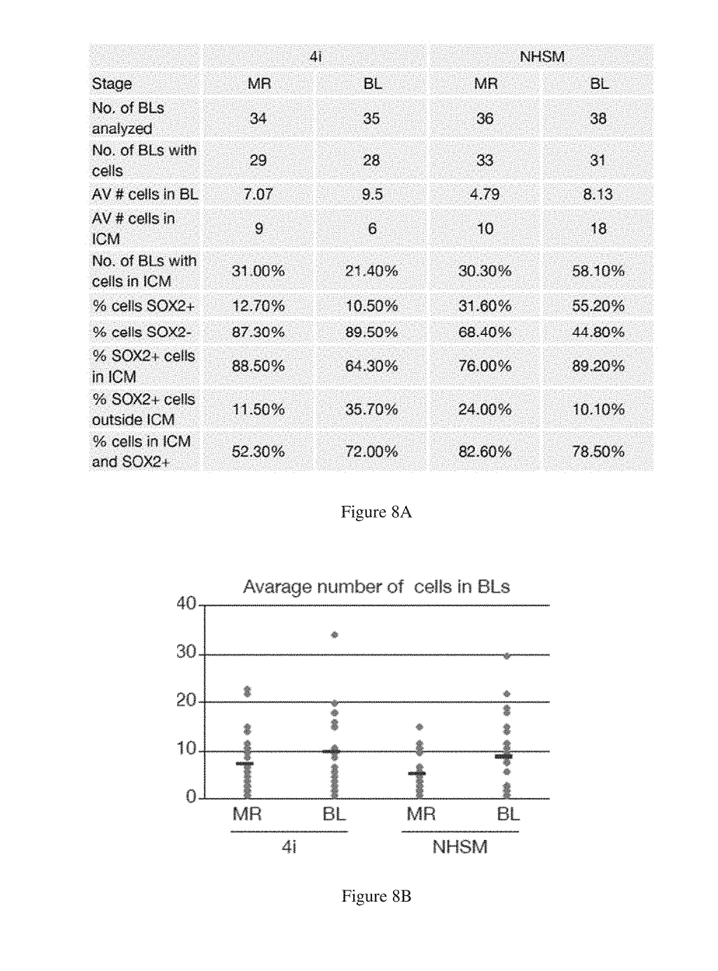

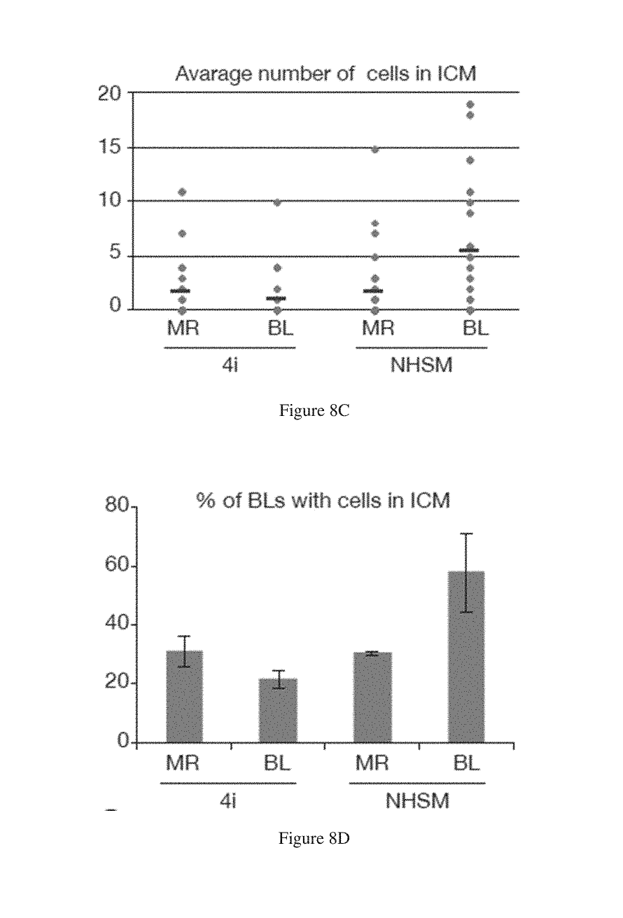

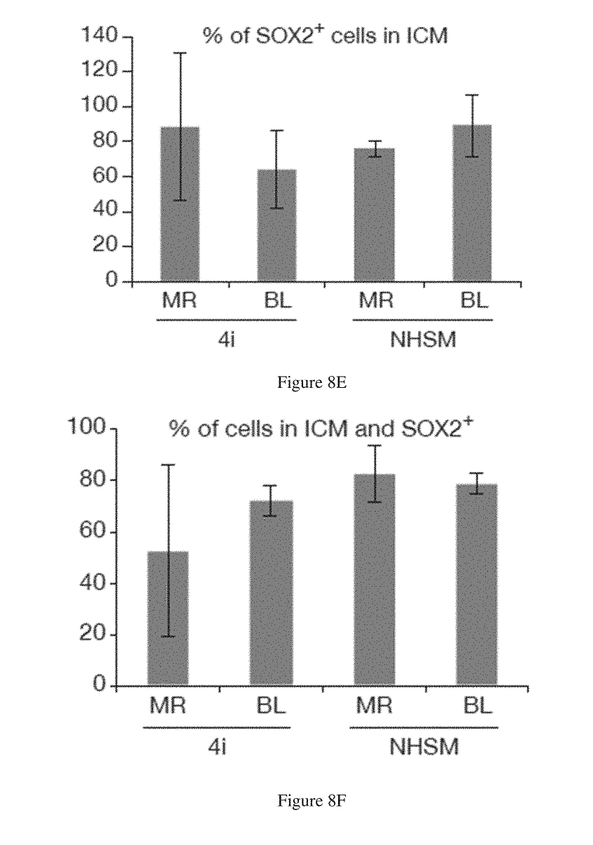

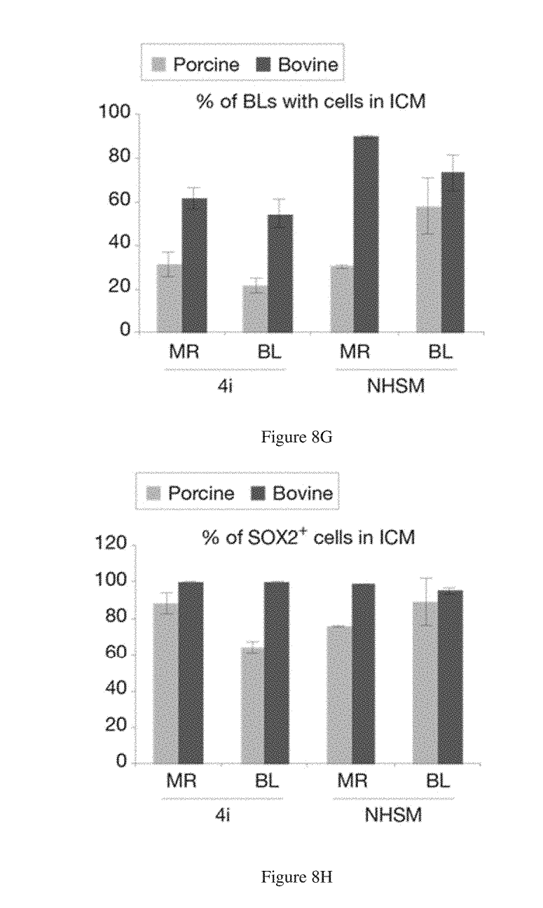

Since injection timing has been shown to play a role in the context of chimeric contribution of mESCs, the 4i and NHSM cultured cells were studied to determine the effects of injection timing in their ability to contribute to a chimeric ICM. To this end, in addition to blastocysts, naïve-like hiPSCs were injected to morula stage bovine and porcine embryos (FIG. 4A). After morula injection, the embryos were cultured for 4 days: the first 4 hours in hiPSC medium followed by 20 hours in mixed medium and an additional 72 hours in embryo culture medium (PZM5 or KSOM) before fixation and further analysis. By comparing morula and blastocyst injections in bovine hosts several noticeable differences were found (FIG. 4B-4G). Although a 1.6-fold increase of total number of cells was found with morula versus blastocyst injection for 4i-hiPSCs, a similar number of cells integrated into the ICM (FIG. 4B). This suggests that 4i-hiPSCs likely survived better or that there was more proliferation (presumably due to the longer culture period) with morula injection. ICM incorporation efficiency, however, was not affected by different injection timing for 4i-hiPSCs (FIG. 4D). Of note is that with morula injection, a majority of the 4i-hiPSCs that incorporated into the bovine ICM lost SOX2 expression (FIG. 4G). Strikingly when NHSM-hiPSCs were injected into morula embryos, an average number of 63.66 cells per blastocyst were found after 4 days of embryo culture and among them 29.62 cells were localized to the ICM (FIG. 4B-4D). As for pig, we observed comparable levels of cell survival and ICM incorporation for 4i-hiPSCs between morula and blastocyst injection (FIG. 4C-4F). NHSM-hiPSCs could be more readily incorporated into the pre-implantation epiblast following blastocyst injection than with morula injection. Again, when comparing the two species similarly improved ICM incorporation efficiency was observed after delivering hiPSCs to bovine morulae (FIG. 8G and FIG. 8H) with only one exception: about half of the ICM incorporated 4i-hiPSCs maintained SOX2 expression in the pig while only 2.6% remained SOX2+ in bovine ICMs after morulae injection (FIG. 8I). A comparison of morula versus blastocyst injection is provided in FIG. 8A, FIG. 8B, FIG. 8C, FIG. 8D, FIG. 8E, FIG. 8F, FIG. 8G, FIG. 8H, and FIG. 8I.

Importantly, the fate of NHSM-hiPSCs after injection to bovine morulae embryos revealed that hPSCs not only engrafted in the bovine embryo but could also proliferate. Immunofluorescence analysis with anti-human and anti-SOX2 antibodies further demonstrated that human cells grew into an ICM-like cluster with partial overlap with bovine ICM. Of note is that this phenomenon was not observed following injection of NHSM-hiPSCs to bovine blastocysts or pig morulae/blastocysts, suggesting that the in vivo niche environments likely differ between species and/or at different stages of pre-implantation development which could impact the fate of donor human cells.

### Example 5

### Interspecies Human-Pig Embryonic Chimeric Formation with Naïve-like hiPSCs

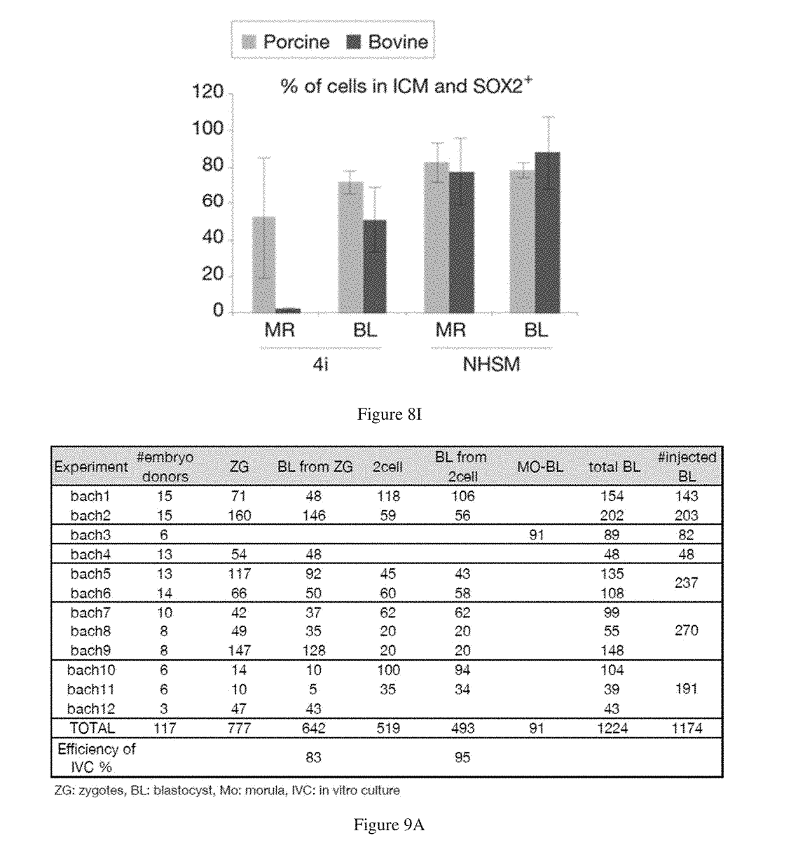

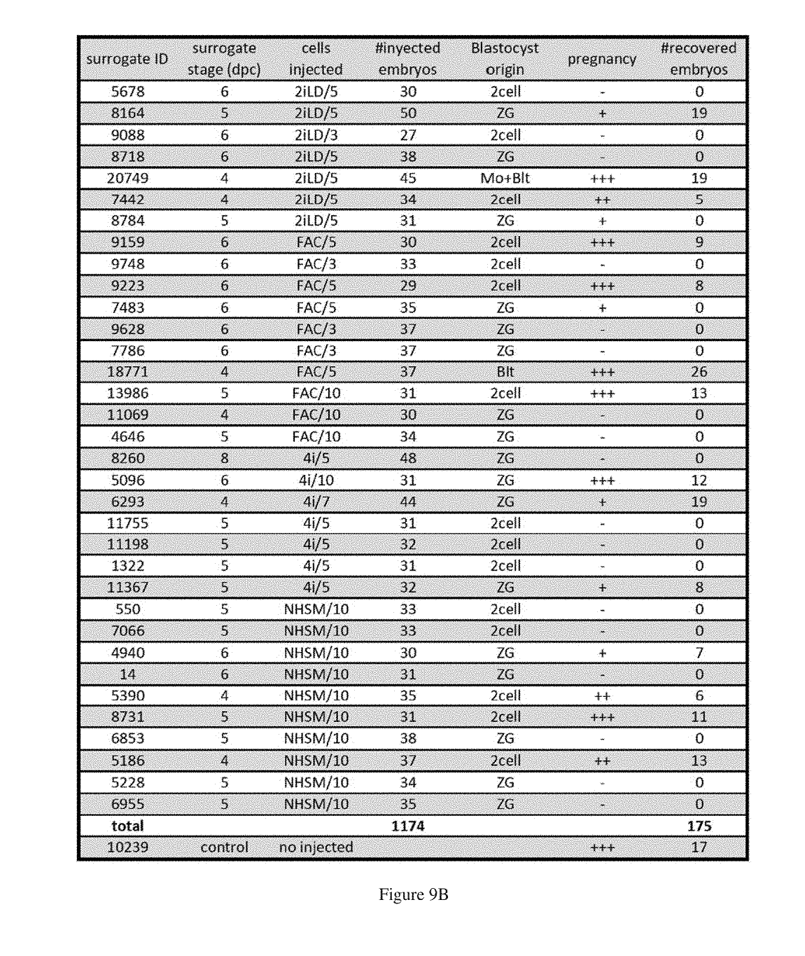

ICM incorporation is informative but not definitive proof for the chimeric-competency of naïve-like hiPSCs. It was studied whether any of the naïve-like hiPSCs could contribute to post-implantation development following embryo transfer. Pig was chosen for this purpose given the higher reproductive rate. To maximize embryo developmental competence we collected in vivo derived embryos that were cultured to the blastocyst stage, injected with hiPSCs and then transferred to synchronized recipient sows. As shown in FIG. 9A, a total of 117 embryo donors were used in this study, from which 777 zygotes, 519 2-cell embryos and 91 morulae were collected. Embryos were temporarily cultured in NCSU-23 medium until they reached the blastocyst stage. Overall, 1,224 good quality blastocysts with a well-defined ICM were selected for subsequent hiPSC injections, of which 642 were derived from zygotes, 493 from 2-cell embryos and 91 from morulae (FIG. 9A).

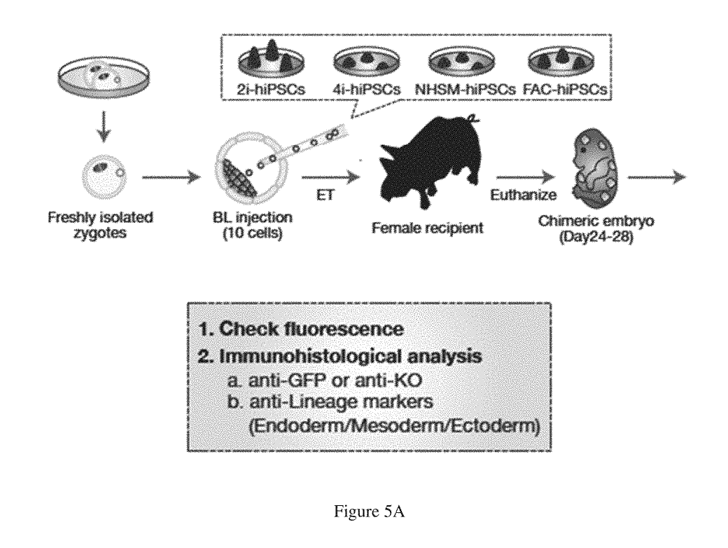

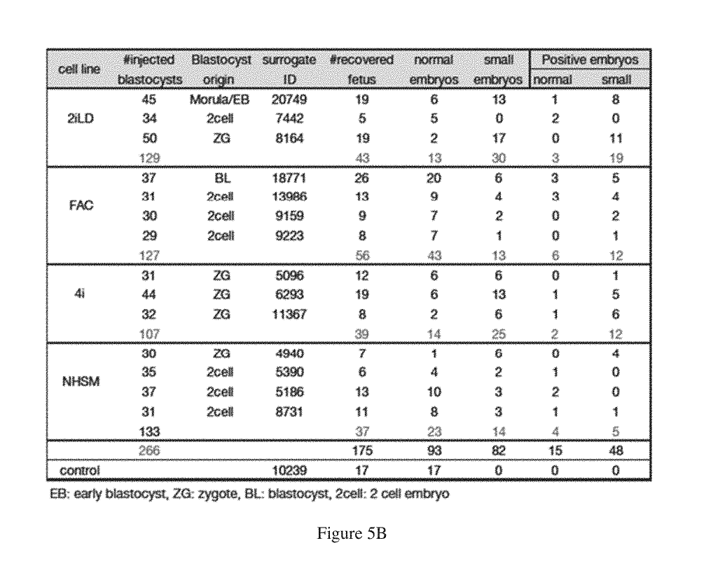

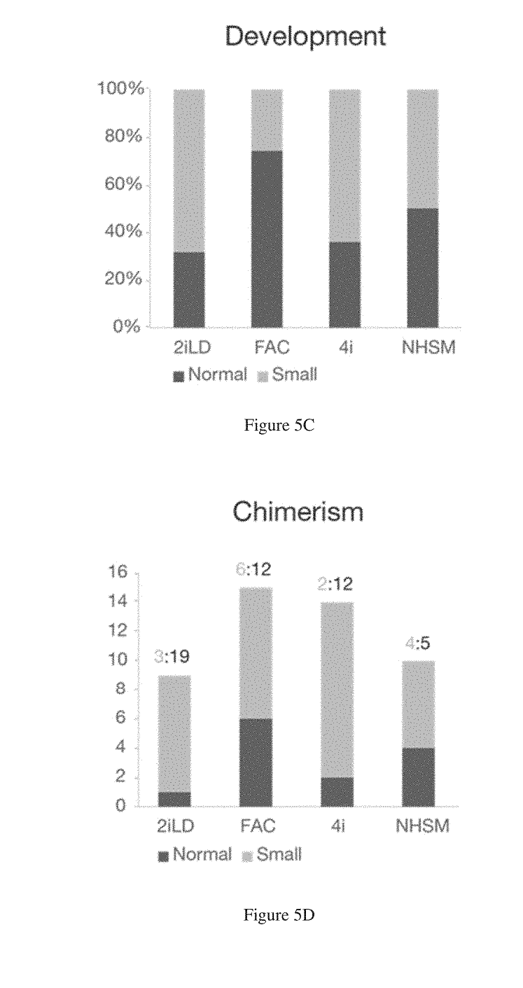

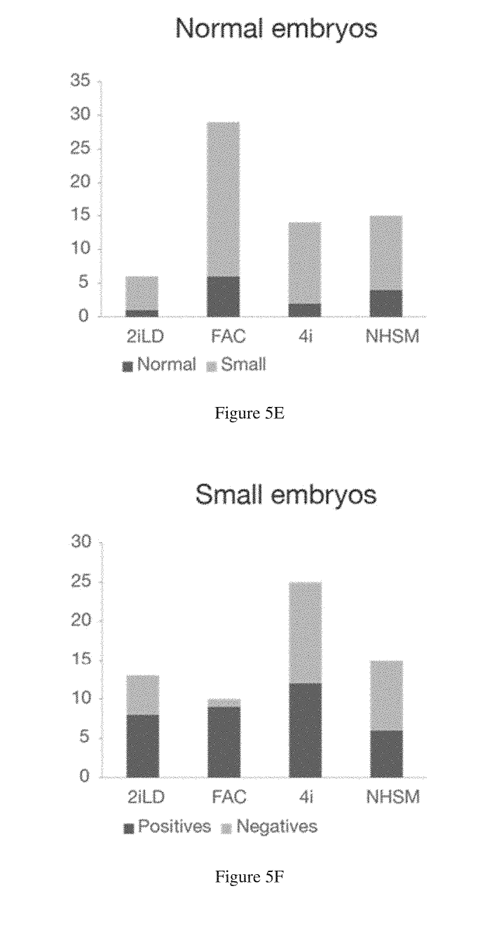

All four types of naïve-like hiPSCs were tested by injecting 3-10 cells into the blastocoel of each blastocyst (FIGS. 5A and 9B). After hiPSC injections, blastocysts were cultured in hiPSC media for 3-4 hours and subsequently changed to mixed medium (NCSU-23 and hiPSC medium mixed at 1:1 ratio) for an additional 20-22 hours. After embryo culture, a total of 1,174 embryos that retained good quality were transferred to surrogate sows. Among 34 surrogate sows that received 30-50 embryos each, 16 were determined to be pregnant upon ultrasonography on day 22-23 of pregnancy (FIG. 9B). Collection of embryos at day 24-28 of development resulted in a total of 175 embryos harvested: 43 from 2iLD-hiPSCs; 56 from FAC-hiPSCs; 39 from 4i-hiPSCs; and 37 from NHSM-hiPSCs (FIG. 5B). In addition, as a control, we collected 17 embryos without hiPSCs injection from one surrogate.

Upon evaluation of the developmental status of obtained embryos it was found that more than half of them manifested retarded growth and were smaller than control embryos (FIG. 5B). When comparing the different cell lines used, it was noticed that embryos injected with FAC-hiPSCs had the most normal-sized embryos while 2iLD-hiPSCs had the fewest, with 4i-hiPSCs and NHSM-hiPSCs somewhere in between (FIG. 5C). From the recovered embryos, and based on fluorescence signals (GFP for 2iLD-hiPSCs and FAC-hiPSCs; KO for 4i-hiPSCs and NHSM-hiPSCs), the presence of human cells was detected in 63 embryos among which 15 appeared to have a normal size an morphology while the rest of the embryos were smaller and/or morphologically underdeveloped (FIG. 5B and FIG. 5D). Closer examination of the smaller embryos showed that 58.5% of them (48 out of 82) had fluorescence signals (FIG. 5B and FIG. 5F). Among all the embryos with positive fluorescence signals the ratio of normal versus small for each cell lines was: 3:19 for 2iLD-hiPSCs; 6:12 for FAC-hiPSCs; 2:12 for 4i-hiPSCs; and 4:5 for NHSM-hiPSCs (FIG. 5D). Among the normal-sized embryos we found 3 positive embryos out of 13 from 2iLD-hiPSCs, 6 out of 43 from FAC-hiPSCs, 2 out of 14 from 4i-hiPSCs and 4 out of 23 from NHSM-hiPSCs (FIG. 5B and FIG. 5E). Notably, all positive embryos found with 2iLD-hiPSCs, 4i-hiPSCs, and NHSM-hiPSCs that were normal-sized showed very limited human cell contributions and in most cases only a few GFP+ or KO+ cells could be detected. Interestingly, it was found that some of the morphologically normal embryos injected with FAC-hiPSCs had relatively higher human cell contribution into different parts of the embryo.

Detection of fluorescence signal alone is not a reliable method to conclude successful human cell contribution, especially in this case where low cell contribution is expected. Thus all embryos deemed positive based on fluorescence signals were processed for histological sectioning and immunohistochemical analyses with anti-GFP or anti-KO antibodies. In accordance with observations based on fluorescence signals, a few cells were detected in limited sections that are KO or GFP positive after staining the normal sized embryos derived from 2iLD-hiPSCs, 4i-hiPSCs, and NHSM-hiPSCs, which precluded successful immunohistochemical analysis with lineage markers. In contrast, more chimeric contribution of FAC-hiPSCs was confirmed via immunohistochemical analysis with GFP antibody, but also proper differentiation of FAC-hiPSCs was observed toward embryonic lineages after immunohistotochemical analyses with antibodies against EPCAM (endoderm), SMA (mesoderm), and CK8 (ectoderm). These results indicate that naïve-like hiPSCs (at least in FAC conditions) could survive and differentiate in post-implantation human-pig chimeric embryos. Interestingly, the outcome of post-implantation chimeric experiments contrasts with the ICM incorporation results for 4i-hiPSCs and NHSM-hiPSCs, suggesting that ICM incorporation alone, although an important indicator, is not sufficient for predicting chimeric-competency of naïve-like hiPSCs, in consistent with mouse studies.

### Example 6

### Discussion

In vivo functional validation through interspecies chimera formation constitutes a parallel and perhaps a more stringent and comprehensive avenue towards defining human naïve pluripotency. By and large, the published studies up to date using the mouse as a host suggest that interspecies chimera formation of human cells into mouse embryos is rather inefficient. It has been argued that a possible cause for this apparent inefficiency may relate to species-specific differences between human and mouse embryogenesis. Therefore, studies utilizing other animal hosts will be helpful in addressing this issue. By focusing on two large animal hosts, pig and cattle, it is shown herein that different types of naïve-like hiPSCs are incorporated at a variable efficiency into pre-implantation host ICMs. Also, naïve-like hiPSCs were able to contribute to the development of post-implantation pig embryos. While still low, the efficiency of post-implantation human-pig chimeric embryo generation appears higher than some reported mouse studies. This could likely be explained by closer timing and rates of pre-implantation development, epiblast morphogenesis during early post-implantation development (disc-shaped epiblast layer in human and pig vs. cup-shaped mouse epiblast) and longer gestation periods between human and pig than between human and mouse. Regardless, however, the examples herein describing the generation of post-implantation chimeric human-pig embryos using naïve-like hiPSCs has proven to be challenging. Election of an evolutionary closer species and/or further improvements in culture conditions may allow for more efficient interspecies chimerism with hPSCs. Despite their low efficiency and low degree of chimeric contribution, newly generated naïve-like hiPSCs grown in FAC medium were able to contribute to chimeric pig post-implantation embryos and intermix with host cells. Immunocytochemistry analyses of the chimeric fetuses further revealed that FAC-hiPSCs not only integrated, but were also able to differentiate as shown by marker expression characteristic of the three germ lineages. To the best of our knowledge, this is the very first report demonstrating successful entry, as well as differentiation, of human cells into the early developmental program of a large animal host.

All hiPSCs of the examples herein survived in the pig and cattle blastocyst environments, integrated into pre-implantation host epiblasts, and expressed the pluripotency marker SOX2, suggesting that these functional features are shared at least among the naïve-like hiPSCs tested. It should be noted, however, that the efficiency and degree of ICM incorporation, as well as the ability to maintain SOX2 expression varied among different lines. Surprisingly, and despite the fact that the use of only one small molecule differs between 4i and NHSM media, 4i-hiPSCs showed a significantly lower percentage of cells remaining undifferentiated (SOX2+) when compared to NHSM-hiPSCs. In addition, both fewer blastocysts and fewer cells showed ICM incorporation with 4i-hiPSCs compared to NHSM-hiPSCs. These observations suggest that subtle changes in culture parameters may endow naïve-like hiPSCs with different features that ultimately may affect their ability for engraftment into pre-implantation pig and cattle embryos. Interestingly, a noticeable fraction of SOX2+ cells was also observed outside of ICMs. This may be either due to precocious neural differentiation of some naïve-like hiPSCs, which remained SOX2+ but could not integrate into pre-implantation epiblasts, or to functional heterogeneity among SOX2+ cells. The ratio of SOX2+ cells detected outside versus inside the ICM may serve as a measure for the degree of compatibility of naïve-like hiPSCs with the in vivo epiblast, a feature that can likely be attributed to specific cell-cell adhesion affinities and extracellular matrix composition that ultimately will determine cell-cell competition variances between host and donor cells.

Morulae versus blastocyst injections, as well as the use of pig and cattle blastocysts, revealed the influence of time and species for donor human cell proliferation and integration. While the porcine blastocyst provides a more permissive environment than morulae, injection of the NHSM-hiPSCs into pre-implantation bovine embryos yielded a contrasting result with more significant human cell proliferation, survival, and integration following morula injection than that observed upon blastocyst injection. Also, when comparing the outcomes in pig versus cattle, bovine embryos seem to serve as a better host for human cells. In this regard, it will be of interest to study the degree of contribution of naïve-like hPSCs to post-implantation bovine embryos. However, this will be a daunting effort considering their much-lower reproductive rate and efficiency. Together, the results herein unveil the fact that distinct in vivo environments at different pre-implantation developmental stages from different species can affect cell survival, proliferation, and chimeric competency of the donor naïve-like hiPSCs. Parameters to be studied that may help explain this diversity include but are not limited to: different pre-implantation developmental strategies; genetic and epigenetic differences, specifically related to the pluripotency regulatory network; and divergent maternal environments. A logic corollary to the observations herein suggests that species with evolutionarily closer preimplantation strategies to human (e.g. non-human primates) may allow for higher chimeric contribution of naïve-like hPSCs.

Divergences in post-implantation development of the host species, in some cases, also have an important role in determining whether successful incorporation of naïve-like hiPSCs in the pre-implantation ICM is subsequently carried on and leads to the formation of a post-implantation chimeric fetus. This is best reflected in experiments in where mouse blastocysts are used as hosts. Although robust ICM incorporation could be observed, naïve-like hPSCs did not survive well in early post-implantation epiblasts, thereby resulting in the low chimeric contribution efficiency observed herein. Observations herein highlight the relevance of cellular heterochrony during development and indicate that spatiotemporally matching of donor hPSCs to the host environment is key for the successful and viable generation of human-mouse chimeric fetuses. With regards to the pig, more chimeric contribution of human cells was observed in growth-retarded fetuses. This observation implies that excessive integration of naïve-like hiPSCs and their subsequent proliferation and differentiation in post-implantation pig embryos, in some cases, have affected normal pig development. Thus, strategies controlling the number of human cells being incorporated into host ICMs may help to improve the degree and efficiency of post-implantation chimera formation. In line with this, although less SOX2+ cells were incorporated into the ICM for FAC condition than 4i and NHSM conditions, the efficiency of obtaining post-implantation chimeric embryos appears higher with FAC, supporting the claim that ICM incorporation alone is not a good predictor for the outcome of post-implantation chimeric embryo formation.

By genetically manipulating the host, an in vivo developmental niche is brought to service, exclusive for donor human cells to populate a certain lineage that subsequently may lead to the formation of a specific tissue and organ (Kobayashi, T., et al. (2010). Generation of rat pancreas in mouse by interspecific blastocyst injection of pluripotent stem cells. Cell 142, 787-799.; Matsunari, H. H., et al. (2013). Blastocyst complementation generates exogenic pancreas in vivo in apancreatic cloned pigs. Proc. Natl. Acad. Sci. U.S.A. 110, 4557-4562; Rashid, T., et al. (2014). Revisiting the flight of Icarus: making human organs from PSCs with large animal chimeras. Cell Stem Cell 15, 406-409; Wu, J., and Izpisua Belmonte, J. C. (2015). Dynamic Pluripotent Stem Cell States and Their Applications. Cell Stem Cell 17, 509-525). This facilitates organ-specific chimeric contribution of human cells but will also help enhance understanding of human embryonic development. Current in vitro differentiation strategies need to overcome various challenges that include the tumor risk posed by undifferentiated hPSCs, the fact that in vitro differentiated cells are mostly immature, and difficulties in generating proper spatial cues to generate tridimensional and functional tissues and organs. The development of efficient interspecies chimera formation methodologies with naïve-like hPSCs constitute an added in vivo strategy for the generation of cells and tissues suitable for transplantation that holds potential to ameliorate the shortage of organ donors worldwide.

### Example 8

### Interspecies Chimeric Complementation

A novel interspecies chimeric complementation approach has been shown to enrich donor cells in certain tissues and organs. Candidate genes were tested in a rat-mouse complementation system. Gene editing of zygotes using the CRISPR/CAS system disables organogenesis of specific lineages by knocking out genes important in the development of a specific organ. For example Pdx1 was inactivated to inhibit formation of the pancreas; Runx-1, Scl/Tal-1, Lmo-2, Tel, and/or Tek were inactivated to inhibit formation of a blood vessel; FAH was inactivated to inhibit formation a liver; Osr1, Lhx1, Pax2, Pax8, Wt1, Hox11, Eya1, Six2, and/or Sal11 were inactivated to inhibit formation of a kidney; Nkx2.5, Tbx5, and/or Mef2c were inactivated to inhibit formation of a heart; Pax6 and/or Rx1 were inactivated to inhibit formation of an eye; Sox9 and/or Scleraxis were inactivated to inhibit formation of cartilage tissue; and Etv2 and/or Prox1 were inactivated to inhibit formation of an endothelial or lymphatic vessel. As a result of said gene inactivation, an empty developmental niche was formed and donor iPSCs from rat were used to fill the niche and an organ was formed enriched for rat cells.

Human iPSCs were also used to enrich human pancreas progenitors in interspecies pig chimeric embryos. To this end, PDX1 was inactivated in a pig zygote by CRISPR/CAS knockout methodology. PDX1 knockout pig zygotes were allowed to develop to a blastocyst then transferred to pseudo-pregnant sows and allowed to develop to day 28. Knockout pig embryos were harvested from euthanized sows and it was found that pancreatic progenitors were absent from the embryos. The procedure was repeated with injection of human iPSCs into the blastocyst and it was found that human iPSCs contributed to early pig development, generated embryonic human-pig chimeras, and colonized emptied pancreatic niches and generated pancreatic progenitors exclusively of human origin in the pig embryos.

### Example 9

### Experimental Procedures

Human iPSC Generation

Conventional primed human iPSCs were generated by reprogramming of human fibroblasts (human foreskin fibroblasts (HFF, ATCC, CRL-2429) with episomal vectors. Briefly, episomal plasmids pCXLE-EGFP (27082), pCXLE-hOCT3/4-shp53-F (27077), pCXLE-hSK (27078), and pCXLE-hUL (27080) were obtained from Addgene. 2×10⁶ HFF fibroblasts were nucleofected with the episomal vectors using 4D-Nucleofector (Program EN150, Lonza) using P2 Primary Cell 4D-Nucleofector kit (Lonza, V4XP). Five days post-nucleofection fibroblasts were replated onto mitotically inactivated MEFs. The next day medium was changed to conventional hPSC culture medium (CDF12), NHSM medium, or FAC medium. Putative iPSC colonies were picked between day 20 and day 30 and transferred to newly prepared MEFs for further cultivation.

Generation and Culture of Naïve-like hiPSCs

Conventional primed hiPSCs were either cultured on plates pre-coated with Matrigel (BD Biosciences) in mTeSR1 medium (Stemcell Technologies) or on MEF in CDF12 medium: DMEM/F12 (Life Technologies, 11330-032), 20% Knockout Serum Replacement (KSR, Life Technologies, 10828), 2 mM Glutamax (Life Technologies, 35050-061), 0.1 mM NEAA (Life Technologies, 11140-050), 0.1 mM β-mercaptoethanol (Gibco, 21985), and 4 ng/ml FGF2 (Peprotech). For the generation of 2iLD-hiPSCs, hiPSCs grown on Matrigel in mTeSR1 medium were pre-treated with 10 uM Y-27632 (Torcris) for 24 hours and then dissociated by TrypLE (Invitrogen). Cells were transduced in suspension with lentiviral vector in the presence of 10 uM Y-27632 and 6 ug/ml polybrene for 1 h. After brief centrifugation, the cells were plated on irradiated DR4 MEF feeders (ATCC) in 2iL media containing 10 uM Y-27632 and 2 ug/ml DOX (Stemgent). 2 iL medium contains: N2B27 medium supplemented with human LIF (10 ng/ml, Peprotech), 3 uM CHIR99021 (Selleckchem) and 1 uM PD035901 (Selleckchem). Three days after transduction, puromycin (1 ug/ml; Invitrogen) was added to the medium. After 7-10 days, dome shaped colonies were manually picked onto fresh MEF feeders and expanded as 2ILD-hiPSCs. NHSM-hiPSCs were directly generated from HFF fibroblasts with episomal vectors using NHSM medium. To prepare 500 ml NHSM medium add 500 ml KnockOut DMEM (Invitrogen), 5 ml Pen-strep (Gibco), 5 ml GlutaMax (Gibco), 5 ml NEAA (Gibco), 5 g AlbumaxI (Invitrogen), 5 ml N2 supplement (Invitrogen; 17502048), L-ascorbic acid 2-phosphate (Sigma), 20 ng/ml human LIF (Peprotech), 20 ng/ml human LR3-IGF1 (Peprotech), 8 g/ml FGF2 (Peprotech), 2 ng/ml TGFβ1 (Peprotech), 3 μM CHIR99021 (Selleckchem), 1 μM PD0325901 (Selleckchem), 5 μM SB203580 (Selleckchem), 5 μM SP600125 (Selleckchem), 5 μM Y27632 (Torcris), and 0.4 μM LDN193189 (Selleckchem). Conversion of NHSM-hiPSCs to 4i-hiPSCs was done by simply changing culture medium from NHSM to 4i. Converted cells were passaged for more 8 times before further analysis and used for subsequent experiments. 4i medium is based on NHSM medium with only the following differences: L-ascorbic acid 2-phosphate, Human LR2-IGF1, and LDN193189 were excluded from 4i medium. TGFβ1 was used at 1 ng/ml and Y27632 at 5 μM. FAC-hiPSCs were generated using HFFs with episomal vectors. FAC medium contains: DMEM/F12 (Invitrogen, 11320) and Neurobasal medium (Invitrogen; 21103) were mixed at 1:1 ratio; N2 supplement (Invitrogen; 17502048); 1×B27 supplement (Invitrogen; 17504044); 2 mM GlutaMax; 1% NEAA; 0.1 mM β-mercaptoethanol (Sigma); 1× Pen-Strep; 50 ug/ml BSA (Sigma); 12 ng/ml FGF2, 10 ng/ml Activin-A (Peprotech) and 3 μM CHIR99021.

Bovine in Vitro Embryo Production

Oocyte recovery and in-vitro maturation (IVM): Ovaries were collected from a slaughterhouse and transported to the laboratory in insulated container filled with pre-warmed saline solution at ˜32° C. The ovaries were washed several times and placed in a water bath at (37° C.) in saline solution for oocyte aspiration. Oocytes were aspirated from 2 to 6 mm antral follicles using a 21 G butterfly needle connected to a vacuum pump. Cumulus-oocyte complexes (COCs) containing compact and complete cumulus cell layers were selected and matured in groups of 50 COCs in 400 μl of M199 medium supplemented with ALA-glutamine (0.1 mM), Na pyruvate (0.2 mM), gentamicin (5 μg/ml), EGF (50 ng/ml), oFSH (50 ng/ml; National Hormone and Peptide Program), bLH (1 ug/ml; National Hormone and Peptide Program), cysteamine (0.1 mM), and 10% fetal bovine serum (FBS; Hyclone, GE Healthcare, South Logan, Utah, USA). IVM was performed for 22-24 hours in a humidified atmosphere of 5% CO2 in air at 38.5° C. In-vitro fertilization (IVF): Fertilization (Day 0) was carried out using frozen-thawed semen. Straws were thawed at 37° C. for 45 seconds and then semen layered onto a 90%/45% Percoll discontinuous density gradient for centrifugation (700×g for 15 minutes) at room temperature. A second centrifugation (300×g for 5 minutes) was performed after discarding the supernatant and re-suspending the spermatozoa pellet in TALP-Sperm (pH=7.4, 295 mOsm) (Parrish, J. J., et al. (1988). Capacitation of bovine sperm by heparin. Biology of reproduction 38, 1171-1180; Parrish, J. J., et al. (1986). Bovine in vitro fertilization with frozen-thawed semen. Theriogenology 25, 591-600). Matured groups of 15-20 COCs were washed twice and placed in 50 μL of fertilization medium. The final sperm concentration was adjusted to 1×10⁶ sperm/ml using a hemocytometer. The fertilization medium was supplemented with BSA (essentially fatty acid free, 6 mg/ml), fructose (90 μg/ml), penicillamine (3 μg/ml), hypotaurine (11 μg/ml), and heparin (20 μg/ml). Oocytes were co-incubated with spermatozoa at 38.5° C. in humidified atmosphere of 5% CO2 in air. Embryo culture (IVC): Presumptive zygotes were mechanically denuded by vortexing for 3-5 minutes in a 1.5 mL tube and 100 uL of SOF-HEPES medium (Holm et al., 1999) and cultured in groups of 15-20 in 50 μL drops of potassium simplex optimized medium supplemented with amino acids and 4 mg/mL of BSA (KSOMaa, pH=7.4, 275 mOsm) (Evolve ZEBV-100, Zenith Biotech, Guilford, Conn., USA) for 7 days. On Day 3, 5% FBS was added. Culture conditions were 38.5° C. in a humidified atmosphere of 5% CO2, 5% O2, and 90% N2. On Days 4 and 7 morulae and blastocysts, respectively, were selected for cell injection.

Porcine Parthenogenetic Embryo Production

Oocyte collection and IVM: Oocytes were aspirated from antral follicles (2-4 diameters) of ovaries from prepubertal gilt ovaries collected at a local slaughterhouse. COCs were washed in TCM-199 (Gibco) containing 0.1% polyvinyl alcohol (PVA), and incubated at 38° C. and 5% CO2 for 48 hours in TCM-199 containing 0.1% PVA, 3.05 mM D-glucose, 0.91 mM sodium pyruvate, 0.5 μg/ml oFSH, 0.5 μg/ml bLH, 10 ng/ml EGF, 10 μg/ml gentamicin (Gibco), and 10% porcine follicle fluid. Parthenogenetic activation: After IVM, maturated oocytes were stripped of their cumulus cells by incubation in 1 mg/ml hyaluronidase and gentle pipetting. Denuded oocytes were washed with MEM containing 25 mM Hepes (Gibco) and electrically activated with two pulses of 120 V/mm for 40 μs, delivered by a BTX Electro Cell Manipulator 2001 (BTX, San Diego, Calif.) in a 0.5 mm chamber containing 0.3 M mannitol, 0.05 mM CaCl2, 0.1 mM MgSO4, and 0.1% bovine serum albumin (BSA). After washing with PZM-5, the oocytes were incubated in the presence of 5 μg/ml cytochalasin B in PZM-5 for 3 hours to prevent second polar body extrusion and thus generate diploid parthenogenetic embryos. Embryo culture: After activation, porcine zygotes were cultured in the 500 μl of PZM-5 (Yoshioka et al., 2012) containing 0.3% BSA for 3-5 days. After 4 days of culture, the culture medium was supplemented with 10% FBS (Gemini Bio-Product, CA) at 38.5° C. in a humidified atmosphere of 5% CO2, 5% O2, and 90% N2.

Microinjection of iPSCs and Embryo Culture

For morula injections, embryos with more than eight blastomeres and before compaction were selected on days 3-4 for pig and day 4 for bovine embryos. For blastocyst injections, embryos with an obvious blastocoel on days 5-6 for the pig and 6-7 for cattle were used. Single cell suspensions were added to a 50 μL drop of cell culture medium containing the embryos to be injected, and placed on an inverted microscope fitted with micromanipulators. Individual cells were collected into a blunt-end micropipette of 20-30 μm internal diameter (ID) connected to a manual hydraulic oil microinjector. Then, the embryo was secured by a holding pipette and a laser system (Saturn 5 Active™, RI) was used to create a whole in the zona pellucida of the embryo. For blastocysts, another laser pulse was applied to the trophectoderm in order to allow access to the blastocoel. Then, the micropipette containing the cells was advanced into the embryo and 10 cells deposited in the blastocoel or periviteline space, for blastocysts and morulae, respectively. Groups of 10-20 embryos were manipulated simultaneously and each session was limited to 40 minutes. Following cell injection, morulae and blastocysts were cultured in the respective cell culture medium for 4 hours, followed by culture for 20 hours in mix medium (1:1) of each cell culture medium and PZM-5 containing 10% FBS (for the porcine embryos) or KSOMaa containing 4 mg/ml BSA (for the bovine embryos). Then, embryos were cultured in PZM-5 containing 10% FBS or KSOMaa containing 4 mg/ml BSA, for pig and cattle embryos respectively, for another 24 hours, except injected bovine morulae that were cultured for 96 hours. At the end of the culture period, GFP or KO signals were observed using an inverted fluorescence microscope (Nikon, Tokyo, Japan) and then embryos were fixed for immunostaining.

Blastocyst Immunostaining

Only blastocyst stage embryos at time of collection were processed for immunostaining as previously described (Ross, P. J., et al. (2008). Polycomb gene expression and histone H3 lysine 27 trimethylation changes during bovine preimplantation development. Reproduction. 136, 777-785). Embryos were washed with phosphate buffered saline (PBS; Gibco) containing 1 mg/mL PVA (PBS-PVA) three times and then fixed in 4% paraformaldehyde containing 1 ml/mL PVA for 15 min at room temperature. After washing three times with PBS-PBA, blastocysts were permeabilized with PBS-PVA containing 1% Triton X-100 for 30 min, washed in PBS-PVA containing 0.1% Triton X-100 (washing buffer; WB), and blocked in PBS-PVA supplemented with 10% normal donkey serum. Embryos were incubated with primary antibodies (rabbit anti-SOX2 antibody, BioGenex, CA, and mouse anti-human nuclei antibody, Millipore) overnight at 4° C. After repeated washes in WB, the embryos were incubated with secondary antibodies (Alexa Fluor 568 anti-rabbit IgG and Alexa Fluor 488 anti-mouse IgG, Invitrogen) at room temperature for 1 h. Then, blastocysts were counterstained with 10 μg/ml Hoechst 33342 at room temperature for 20 min. Stained blastocysts were mounted on a glass slide containing ProLong Gold antifade solution (Invitrogen), covered by a coverslip, and imaged using an inverted fluorescence microscope.

In Vivo Embryo Recovery and Transfer

Animals: This work was conducted in a commercial pig farm located in Southeastern Spain (Murcia, Spain), in the pig experimental unit of the University of Murcia (Murcia, Spain). Weaned crossbred sows (Landrace×Large-White) from the same genetic line (2-6 parity) were used as embryo donors and recipients. The sows were kept individually in crates in a mechanically ventilated confinement facility. The semen donors were sexually mature boars (2-3 years of age) housed in climate- controlled individual pens (20-25° C.) at a commercial insemination station in Murcia (Spain) Animals had ad libitum access to water and were fed commercial diets according to their nutritional requirements. All the experimental procedures used in this study were performed in accordance with Directive 2010/63/EU EEC for animal experiments and were reviewed and approved by the Ethical Committee for Experimentation with Animals (code: 69/2014), the Research Ethics Committee (code: 1086/2015), and the Biosafety Committee of the University of Murcia, Spain; the Murcia Regional Ministry of Agriculture and Water (code: 273.705); and the Murcia Regional Ministry of Health (code: 061015), Spain.

Superovulation and detection of estrus: Weaning was used to synchronize the estrus in donors and recipients. Only sows with a weaning-to-estrus interval of 3-4 days were selected as donors and recipients. The superovulation of donors was induced by the intramuscular administration of 1000 IU equine chorionic gonadotropin (eCG; Foligon, Intervet, Boxmeer, The Netherlands) 24 h after weaning. Estrus was checked twice per day by exposing sows to a mature boar (nose-to-nose contact) and applying manual back pressure. Females that exhibited a standing estrous reflex were considered to be in estrus. Only sows with clear signs of estrus at 48-72 h post-eCG administration were further intramuscularly administered with 750 IU of human chorionic gonadotropin (Veterin Corion, Divasa, Farmavic S.A., Barcelona, Spain) at the onset of estrus.

Insemination of donors: The donors were post-cervically inseminated at 6 and 24 h after the onset of estrus. The insemination doses (1.5×10⁹ spermatozoa in 45 mL) were prepared from sperm-rich fractions of the ejaculates extended in Beltsville thawing solution extender (Pursel and Johnson, 1975) and were stored for a maximum of 72 h at 18° C.

Embryo recovery and evaluation: The collection of embryos was performed in a specifically designed surgical room located on the farm. The donors were subjected to a mid-ventral laparotomy on Day 2 of the estrous cycle (Day 0: onset of estrus). The donors were sedated with azaperone (2 mg/kg body weight, intramuscular). General anesthesia was induced using sodium thiopental (7 mg/kg body weight, intravenous) and was maintained with isoflurane (3.5-5%). After exposure of the genital tract, the corpora lutea on the ovaries were counted. Zygotes were collected by flushing each oviduct with 20 mL of protein-free embryo recovery medium consisting of Tyrode's lactate (TL)-HEPES-polyvinyl alcohol (PVA)-medium (TL-HEPES-PVA) (Funahashi, H., et al. (2000). Zona reaction in porcine oocytes fertilized in vivo and in vitro as seen with scanning electron microscopy. Biology of reproduction 63, 1437-1442) with some modifications (Martinez, E. A., et al. (2014). Successful non-surgical deep uterine transfer of porcine morulae after 24 hour culture in a chemically defined medium. PLoS One 9, e104696). Collected embryos were washed three times in TL-HEPES-PVA, placed in sterile Eppendorf tubes containing 1.5 mL of the same medium and transported in a thermostatically controlled incubator at 39° C. to the laboratory at the University of Murcia within 1 h after collection. Embryos were then evaluated for morphology under a stereomicroscope at a magnification of 60×. Only zygotes with a single cell and two visible polar bodies were used in the experiments.

In vitro embryo culture and assessment of in vitro embryo development: After injections, zygotes were then transferred (40 zygotes per well) to a 4-well multidish (Nunc) containing 500 μL of glucose-free NCSU-23 medium (Petters, R. M., and Wells, K. D. (1993). Culture of pig embryos. J Reprod Fertil Suppl 48, 61-73) that was supplemented with 0.3 mM pyruvate and 4.5 mM lactate for 24 h and then changed to fresh NCSU-23 medium containing 5.5 mM glucose for an additional 5 days. Cultures were performed at 39° C., 5% CO2 in air and 95-97% relative humidity. At Day 5, embryo culture wells were supplemented with 10% (v/v) fetal calf serum (FCS). In vitro embryo development was evaluated under a stereomicroscope at 24 h and 6 days of culture to determine cleavage and blastocyst formation rates, respectively. An embryo that had cleaved to the 2- to 4-cell stage was defined as cleaved, and an embryo with a well-defined blastocoel and an inner cell mass and trophoblast totally discernible was defined as a blastocyst. The cleavage rate was defined as the percentage of oocytes that had divided to the 2- to 4-cell stage. Blastocyst formation was the percentage of 2- to 4-cell cell embryos that developed to the blastocyst stage. The total efficiency was described as the percentage of the total number of cultured oocytes that reached the blastocyst stage. Blastocysts were injected as described above.

Culture of hiPSC injected blastocysts: Immediately after hiPSC injection, the blastocysts were incubated in 500 μL of medium used for the culture of hiPSCs for 3-4 h and then changed to NCSU-23 medium and hiPSC medium (1:1) for an additional 20-22 h. Surgical embryo transfer. Injected blastocysts were loaded into a Gynetics embryo transfer catheter (Gynetics Medical Products N.V., Lomel, Belgium) connected to a 1mL syringe for transfer into the recipients. The embryo transfer medium was NCSU-23 supplemented with 10 mM HEPES, 0.4% (v/v) BSA and 10% (v/v) FCS. The embryo transfer catheter was loaded with air bubbles that separated the 30-μL drop of medium that contained the embryo from two drops of medium before and after the embryo. All transfers were conducted in asynchronous (−24 hours to embryo collection) recipients. One hour before the transfer, each recipient received a single intramuscular injection of a long-acting amoxicillin suspension (Clamoxyl LA; Pfizer, Madrid, Spain) at a dose of 15 mg/kg. The transfers were conducted using the same procedure described previously for embryo collection. The embryos were transferred to the tip of a uterine horn (15 to 20 cm from the uterotubal junction) with the embryo transfer catheter inserted through the uterine wall, which was previously punctured with a blunt Adson forceps. Post transfer, all recipients were evaluated daily for behavioral changes, including signs of estrus beginning at 12 days post ET. Pregnancy was diagnosed by transabdominal ultrasonography (Logiq Book XP, General Electric, Solingen, Germany) on Days 20 to 22 post-transfer. All pregnant sows were deeply anesthetized on days 23 to 25 post-transfer and subsequently euthanized by using a captive bolt pistol. Immediately, a midline longitudinal incision was made between the posterior pair of nipples and the ovaries and uterus were located. The cervix and the ovarian stalks were occluded with transfixing ligatures and the reproductive tract removed. The genital tract was then placed in water tight plastic bags kept on ice and transported to the laboratory within 20 min. Once in the laboratory the uterus was opened and fetuses removed from the placenta tissues and numbered in sequential order. Fetuses were individually measured and weighed. Afterwards, each fetus was checked for fluorescence emission using a epifluorescence stereomicroscope (Nikon SMZ 18, Japan).

Immunocytochemistry

At days 24-28 of gestation, surrogates were euthanized and embryos were dissected and immerse in paraformaldehyde during 4 hours for small-sized embryos and overnight for normal-sized embryos. After overnight cryoprotection in 30% sucrose solution (Sigma), the bifurcations were embedded in OCT compound (Sakura Finetek) and frozen in dry ice. Sections (10 μm thick for small-sized embryos and 20 μm for normal-sized embryos) of the different embryos were cut on a Leica cryostat. For immunohistochemistry, we used standard staining procedures and antigen retrieval solution (HistoVT one, Nacali Tesque, INC) according to manufacturer's instructions. The primary antibodies used were rabbit anti-monomeric Kusabira-Orange 2 (MBL, 1:500), rabbit anti-GFP (MBL, 1:500), rat anti-cytokeratin 8 (TROMA-I, DSHB, 1:20), mouse IgG1 anti-epithelial antigen (Dako, 1:200), mouse IgG2a anti α-smooth muscle antibody (Sigma, 1:200).

Statistical Analyses

Human cell contribution data was analyzed by Tukey's honestly significant difference test using an R.A. probability of P<0.05 to indicate significant differences.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-23. (canceled)

24. A mammalian organ or tissue isolated from a chimeric mammal comprising cells derived from at least a first mammal and a second mammal, wherein the organ or tissue is derived from an induced pluripotent stem cell (iPSC) cell from the second mammal, wherein the first and second mammal are different species.

25. The mammalian organ or tissue of claim 24, wherein the organ or tissue comprises at least one of the group consisting of liver, kidney, pancreas, hematopoietic stem cells, spleen, bone marrow, heart, lung, skin, cornea, eye, spinal cord, uterus, intestine, heart valve, bone, cartilage, tendon, ligament, lymphatic vessel, and blood vessel.

26. The mammalian organ or tissue of any one of claim 24, wherein the second mammal is selected from the group consisting of mouse, rat, rabbit, guinea pig, human, cow, pig, horse, goat, and sheep.

27. (canceled)

28. The mammalian organ or tissue of claim 24, wherein the first mammal is selected from the group consisting of mouse, rat, rabbit, guinea pig, cow, pig, horse, goat, and sheep.

29. (canceled)

30. The mammalian organ or tissue of claim 24, wherein cells of the first mammal comprise a genetic modification at one or more loci, wherein the genetic modification inactivates at least one gene selected from the group consisting of FAH, NKX2.5, TBX5, MEF2C, Osr1, Lhx1, Pax2, Pax8, Wt1, Hox11, Eya1, Six2, Sal11, Etv2, Trox1, Ronx-1, Scl/Tal-1, Lmo-2, Tel, Tek, Sox9, Scleraxis, Pax6, and Rx.

31.-33. (canceled)

34. The mammalian organ of claim 24, wherein the first mammal comprises a genetic modification that inactivates Pdx1 and the cells from the second mammal form at least a portion of a pancreas.

35. The mammalian organ of claim 24, wherein the first mammal comprises genetic modifications that inactivate Runx-1, Scl/Tal-1, Lmo-2, Tel, and/or Tek and the cells from the second mammal form at least a portion of a blood vessel or hematopoietic cell.

36. The mammalian organ of claim 24, wherein the first mammal comprises genetic modifications that inactivate FAH and the cells from the second mammal form at least a portion of a liver.

37. The mammalian organ of claim 24, wherein the first mammal comprises genetic modifications that inactivate Osr1, Lhx1, Pax2, Pax8, Wt1, Hox11, Eya1, Six2, and/or Sal11 and the cells from the second mammal form at least a portion of a kidney.

38. The mammalian organ of claim 24, wherein the first mammal comprises genetic modifications that inactivate Nkx2.5, Tbx5, and/or Mef2c and the cells from the second mammal form at least a portion of a heart.

39. The mammalian organ of claim 24, wherein the first mammal comprises genetic modifications that inactivate Pax6 and/or Rx and the cells from the second mammal form at least a portion of an eye.

40. The mammalian organ of claim 24, wherein the first mammal comprises genetic modifications that inactivate Sox9 and/or Scleraxis and the cells from the second mammal form at least a portion of a cartilage tissue.

41. The mammalian organ of claim 24, wherein the first mammal comprises genetic modifications that inactivate Etv2 and/or Prox1 and the cells from the second mammal form at least a portion of an endothelial or lymphatic vessel.

42.-69. (canceled)

70. A method of making an induced pluripotent stem cell (iPSC) comprising:
a) transfecting a population of human foreskin fibroblasts (HFF) with at least three episomal vectors comprising pCXLE-hOCT3/4-shp53-F, pCXLE-hSK, and pCXLE-hUL;
b) contacting the transfected population of HFFs with a population of mitotically inactivated mouse embryonic fibroblasts (MEFs);
c) contacting the population of HFFs and the population of MEFs with an FAC medium comprising a base media, an FGF, an Activin, and a WNT activator until at least one colony comprising an iPSC has formed; and
d) transferring the colony comprising an iPSC to a fresh population of mitotically inactivated MEFs.

71. The method of claim 70, wherein the base media comprises DMEM/F12, Neurobasal medium, N2 supplement, B27 supplement, GlutaMax, non-essential amino acids, beta-mercaptoethanol, and antibiotics.

72. The method of claim 70, wherein the FGF comprises an FGF2.

73. The method of claim 70, wherein the Activin comprises an Activin A.

74. The method of claim 70, wherein the WNT activator comprises a GSK inhibitor.

75. (canceled)

76. The method of claim 70, wherein the HFF are transfected with pCXLE-EGFP.

77. The method of claim 70, wherein the FAC medium comprises bovine serum albumin
